(19) United States
(12) Patent Application Publication
(10) Pub. No.: US 2026/0308547 A1
(43) Pub. Date: Oct. 8, 2026

(54) GENE THERAPY FOR LYSOSOMAL ACID LIPASE DEFICIENCY (LAL-D)

(71) Applicant: RESEARCH INSTITUTE AT NATION WIDE CHILDREN'S HOSPITAL, Columbus, OH (US)

(72) Inventor: Paul Taylor Martin, Bexley, OH (US)

(21) Appl. No.: 19/490,185

(22) PCT Filed: Jun. 6, 2024
(86) PCT No.: PCT/US2024/032823

§ 371 Date: Dec. 4, 2025

Related U.S. Application Data
(60) Provisional application No. 63/471,616, filed on Jun. 7, 2023.

(51) Int. Cl. C12N 9/20; A61K 9/00; A61K 38/00; A61K 48/00; A61P 3/00; C12N 15/86

(52) U.S. Cl. CPC C12N 9/20; A61K 9/0019; A61K 48/0058; A61P 3/00; C12N 15/86; A61K 38/00; C12N 2750/14143; C12N 2830/008; C12Y 301/01003

(57) ABSTRACT

The disclosure provides gene therapy vectors, such as adeno-associated virus (AAV), designed for treatment of Lysosomal Acid Lipase Deficiency (LAL-D) disorders, such as Wolman disease and cholesteryl ester storage disease (CESD), nonalcoholic fatty liver disease (NAFLD), or nonalcoholic steatohepatitis (NASH). The disclosed rAAV provide a wild type lipase A (LIPA) cDNA to a subject in need which results in expression of the wild type protein.

### CROSS REFERENCE TO RELATED APPLICATIONS

This application claims priority benefit of U.S. Provisional Application No. 63/471,616, filed on Jun. 7, 2023, which is incorporated herein by reference in its entirety.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 59257_SeqListing.xml; Size: 26,068 bytes; Created: Jun. 3, 2024.

### FIELD OF THE INVENTION

The disclosure provides gene therapy vectors, such as adeno-associated virus (AAV), designed for treatment of Lysosomal Acid Lipase Deficiency (LAL-D) disorders, such as Wolman disease and cholesteryl ester storage disease (CESD), and disorder related to lipid storage or accumulation, such as nonalcoholic fatty liver disease (NAFLD) or nonalcoholic steatohepatitis (NASH). The disclosed rAAV provide a wild type human lipase A (LIPA) cDNA under the control of a liver-specific promoter, such as LP1, to a subject in need which results in expression of the wild type human LAL protein.

### BACKGROUND

Lysosomal Acid Lipase Deficiency, LAL-D, is a lysosomal storage disorder caused by recessive mutations in the Lipase A (LIPA) gene that result in a failure of the lysosomal acid lipase (LAL) protein to sufficiently hydrolyze cholesterol esters into free cholesterol and triglycerides into free fatty acids in the lysosome. LAL occupies a critical and essential position in the control of plasma lipoprotein levels and in the prevention of cellular lipid overload, especially in the liver and spleen (Li et al., Arterioscler Thromb Vasc Biol 39: 850-856, 2019; Aguisanda et al. Curr Chem Genom Trans/Med 11: 1-18, 2017). The LIPA gene is the only gene with this lysosomal function in the human genome. LAL-D is a rare genetic disease, with prevalence ranging from 1 in 40,000 to 1 in 300,000, though disease incidence may be underestimated through failed diagnosis in some instances (Pastores et al., Lysosomal Acid Lipase Deficiency: Therapeutic Options. Drug Des Devel Ther 14: 591-601, 2020).

Null LIPA gene mutations cause Wolman disease (WD), a fatal disease of infancy named after Moshe Wolman, who reported one of the first cases (Abromov et al., AMA J Dis Child 91: 282-286, 1956). WD is characterized by hepatomegaly with liver dysfunction, dyslipidemia (elevated serum triglycerides and LDL-cholesterol with reduced HDL-cholesterol), hepatosplenomegaly, pulmonary fibrosis, and adrenal calcification and insufficiency. Infants manifest disease in the first month of life and fail to thrive, most likely due to liver disease combined with a failure to absorb nutrients through the intestinal lining. Median lifespan of untreated WD infants is 3.7 months. Partial loss of function LIPA mutations, usually with 1-12% of normal activity, give rise to cholesteryl ester storage disease (CESD), a later onset, less severe disease form. While CESD need not result in premature death, it is associated with significant morbidity, including liver fibrosis and cirrhosis (and also liver failure). Chronic dyslipidemia in LAL-D may also cause accelerated atherosclerosis and high risk of cardiac disease, including myocardial infarction, and cerebrovascular complications, including stroke. Liver biopsy in LAL-D patients typically demonstrate micro- and macro-vascular steatosis involving Kuppfer cells and hepatocytes, accompanied by fibrosis and cirrhosis as the disease progresses. Unlike other lysosomal storage disorders such as Gaucher disease and Niemann-Pick disease, there appears to be no primary CNS involvement (though histological studies are lacking).

While LAL-D is a rare genetic disorder, the pathology findings in LAL-D speak to larger and far more common significant health issues that are found in the general population. For example, reduced LAL-D activity is a biomarker for nonalcoholic fatty liver disease (NAFLD), a disorder affecting many millions of American adults and children. Such reductions in LAL activity are progressive with the increasing severity of liver disease; LIPA enzyme activity decreases from simple liver steatosis to nonalcoholic steatohepatitis (NASH) to cryptogenic liver cirrhosis. Additionally, there is a LIPA haplotype strongly associated with coronary artery disease. See Du, H, and Grabowski, GA (2004). Lysosomal acid lipase and atherosclerosis. Curr Opin Lipidol 15: 539-544; and Wild, P S, Zeller, T, Schillert, A, Szymczak, S, Sinning, C R, Deiseroth, A, et al. (2011). A genome-wide association study identifies LIPA as a susceptibility gene for coronary artery disease. Circ Cardiovasc Genet 4: 403-412. The buildup of fatty acids in LAL-D mimics human conditions such as morbid obesity and obesity related to type II diabetes. Given how easily AAV transduces the liver, and how important the liver is in controlling lipoprotein-based metabolism, it is conceivable that LIPA gene therapy could be applied to these other genetic, and even non-genetic, human diseases related to obesity, based on the relationship of LAL-D to fat absorption in these other disorders.

### SUMMARY

The disclosure provides for a clinical AAV vector used in gene replacement therapy for LAL-D including those disorders caused by mutations in the LIPA gene and non-genetic disorders that are associated with lipid accumulation and storage.

In one aspect, described herein is a polynucleotide comprising (a) one or more liver-specific regulatory control elements and (b) LIPA cDNA sequence. In some embodiments, the regulatory control element is a liver specific LP1 promoter comprising a nucleotide sequence set forth in SEQ ID NO: 3, or fragments thereof which retain regulatory control or promoter activity. In some embodiments, the vector comprises a late SV40 poly adenylation sequence. In some embodiments, the LIPA cDNA is the LIPA variant 1 cDNA, and the LIPA cDNA comprises the polynucleotide sequence set forth in SEQ ID NO: 1.

In one embodiment, the disclosure provides for a rAAV comprising a nucleotide sequence that encodes a functional lysosomal acid lipase (LAL) protein, wherein the nucleotide has, e.g., at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98%, 99%, or 100% sequence identity to SEQ ID NO: 1, wherein the protein retains LAL activity, such as the activity to hydrolyze cholesterol esters into free cholesterol and triglycerides into free fatty acids in the lysosome. For example, the nucleotide sequence that encodes a functional LAL protein may comprise one or more base pair substitutions, deletions or insertions which do affect the function of the LIPA protein. Furthermore, the nucleotide sequence that encodes a functional LIPA protein may comprise one or more base pair substitutions, deletions or insertions may increase or reduce expression of the LAL protein, and this change in expression pattern may be desired for treatment of the LAL-D or the disorder related to lipid storage and accumulation.

In another embodiment, the disclosure provides for a rAAV comprising a nucleotide sequence that encodes a functional LAL protein, wherein the protein comprises an amino acid sequence that has, e.g., at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98%, 99%, or 100% sequence identity to SEQ ID NO: 2, wherein the protein retains LAL activity, such as the activity to hydrolyze cholesterol esters into free cholesterol and triglycerides into fatty acids in the lysosome. For example, the nucleotide sequence that encodes a functional LAL protein may comprise one or more amino acid substitutions, deletions, or insertions which do affect the function of the LAL protein.

The term “sequence identity”, “percent sequence identity”, or “percent identical” in the context of nucleic acid or amino acid sequences refers to the residues in the two sequences which are the same when aligned for maximum correspondence. The length of sequence identity comparison may be over the full-length of the genome, the full-length of a gene coding sequence, or a fragment of at least about 500 to 5000 nucleotides, is desired. However, identity among smaller fragments, e.g., of at least about nine nucleotides, usually at least about 20 to 24 nucleotides, at least about 28 to 32 nucleotides, at least about 36 or more nucleotides, may also be desired. The percent identity of the sequences can be determined by techniques known in the art. For example, homology can be determined by a direct comparison of the sequence information between two polypeptide molecules by aligning the sequence information and using readily available computer programs such as ALIGN, ClustalW2, and BLAST. In one embodiment, when BLAST is used as the alignment tool, the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR.

In another aspect, the disclosure provides for an rAAV construct contained in the plasmid comprising the nucleotide sequence of SEQ ID NO: 4. For example, the rscAAV.LP1.LIPA vector comprises the nucleotide sequence within and inclusive of the ITR's of SEQ ID NO: 4. The rAAV vector comprises the 5′ ITR, LP1 promoter, the coding sequence for the human LIPA gene, SV40 late polyA, and 3′ ITR. In this embodiment, the 3′ ITR contains a deletion of the terminal resolution site (dTR), which inhibits Rep protein nicking of the single stranded viral genome. The presence of the dTR in the 3′ ITR increases self-complementary binding of the viral genome to itself, which it may do because of its small (2.2 kB) size that allows for a double-stranded viral genome to be packaged within the viral capsid. The self-complementary nature of rscAAVrh74.LP1.LIPA facilitates both the speed and the extent of gene expression relative to constructs that remain as a single-stranded viral genome. In one embodiment, the vector comprises nucleotides 1853-4094 of SEQ ID NO: 4. The nucleotides within the ITRs may be in forward or reverse orientation. For example, the LP1 promoter sequence, human LIPA gene sequence, and SV40 late polyA sequence and may be in forward or reverse orientation. In another embodiment, the vector comprises a nucleotide sequence that has about at least 85%, 86%, 87%, 88%, 89%, 90%, 91%, 92%, 93%, 94% 95%, 96%, 97%, 98%, 99%, or 100% sequence identity to the nucleotides of 1853-4094 of SEQ ID NO: 4. The plasmid set forth in SEQ ID NO 4 further comprises kanamycin resistance and an origin of replication.

In another aspect, described herein is a recombinant adeno-associated virus (rAAV) having a genome comprising a polynucleotide sequence described herein. In some embodiments, the rAAV is of the serotype AAV1, AAV2, AAV3, AAV4, AAV5, AAV6, AAV7, AAV8, AAV9, AAVRH10, AAVrh74, AAVrh, AAV11, AAV12, AAV13, Anc80, AAV7m8, or their derivatives.

In some embodiments, the genome of the rAAV comprises an LP1 promoter and LIPA cDNA. An exemplary genome comprises the LP1 promoter, and the LIPA cDNA such as the rscAAVrh74.LP1.LIPA, the rAAV set out as nucleotides 1853-4094 of SEQ ID NO: 4. The LP1 promoter contains the core liver-specific elements of the HCR (human alipoprotein E/C-gene locus control region) and human a1 anti-trypsin (hAAT) promoter as set out in SEQ ID NO: 3. The LP1 promoter allows for AAV packaging of the self-complementary double-stranded viral genome, which is not allowed with promoters that are of a larger size.

In another aspect, described herein is an rAAV particle comprising an rAAV described herein.

Compositions comprising any of the rAAV described herein or any of the viral particles described herein. The disclosed composition may be formulated for any means of delivery, such as direct injection into the cerebrospinal fluid, intracerebroventricular delivery, intrathecal delivery, intraperitoneal delivery, intraarterial delivery, or intravenous delivery.

In addition, the disclosed composition is formulated for intravenous delivery or intraperitoneal delivery and comprises a dose of rAAV or rAAV particles of about 1 e12 vg/kg to about 8×10¹³ vg/kg.

Methods of treating LAL-D or a disorder related to lipid storage or accumulation in a subject in need thereof comprising administering a polynucleotide, an rAAV, or an rAAV particle described herein are specifically contemplated. In some embodiments, the methods further comprise administering an immunosuppressing agent prior to, after, or simultaneously with the polynucleotide, rAAV, or rAAV particle. The LAL-D includes a disorder or disease caused by a mutation in the LIPA gene, such as Wolman disease or cholesteryl ester storage disease (CESD). The disorder related to lipid storage or accumulation include coronary artery disease, atherosclerosis, type II diabetes, obesity, or nonalcoholic fatty liver disease (NAFLD). Treatment of LAL-D or a disorder related to lipid storage of accumulation in a subject may include reduction of lipid or triglyceride content in the liver of the subject and/or increasing or extending survival of the subject.

The disclosure also provides for methods of treating dyslipidemia or hypercholesterolemia in a subject in need thereof comprising administering a polynucleotide, an rAAV or an rAAV particle described herein are specifically contemplated. In some embodiments, the methods further comprise administering an immunosuppressing agent prior to, after, or simultaneously with the polynucleotide, rAAV, or rAAV particle.

The disclosure also provides for method of decreasing triglycerides, cholesterol, and/or fatty acids in a subject in need thereof comprising administering a polynucleotide, an rAAV, or an rAAV particle described herein are specifically contemplated. In some embodiments, the methods further comprise administering an immunosuppressing agent prior to, after, or simultaneously with the polynucleotide, rAAV, or rAAV particle.

In any of the disclosed methods, the polynucleotide, rAAV, rAAV particle, or composition are intravenously delivered to the subject. In some embodiments, the method further comprises a step of administering an immunosuppressing agent. For example, the polynucleotide, rAAV, rAAV particle, or composition is administered simultaneously, prior to, or after administration of an immunosuppressing agent, such as prednisone, prednisolone, rapamycin, methotrexate, myophenolate mofetil, tacrolimus, mycophenolate, or rituximab. In any of the methods, the subject has a mutation in the LIPA gene. These mutations include those currently known, such as those set out in Table 1 herein, or a mutation(s) in the LIPA gene identified in the future that is associated with LAL-D.

A “subject,” as used herein, can be any animal, and may also be referred to as the patient. Preferably the subject is a vertebrate animal, and more preferably the subject is a mammal, such as a domesticated farm animal (e.g., cow, horse, goat, pig) or pet (e.g., dog, cat, hamster, chinchilla). In some embodiments, the subject is a human. In some embodiments, the subject is a pediatric subject. In some embodiments, the subject is a pediatric subject, such as a subject ranging in age from 1 to 10 years or the subject is an infant ranging in age for one month to 12 months. In some embodiments, the subject is 4 to 15 years of age. The subject, in one embodiment, is an adolescent subject, such as a subject ranging in age from 10 to 19 years. In other embodiments, the subject is an adult (e.g., 18 years or older).

In another aspect, described herein is the use of a polynucleotide, an rAAV, or an rAAV particle described herein in the preparation of a medicament for the treatment of an LAL-D or a disorder related to lipid storage or accumulation. In some embodiments, the LAL-D is Wolman disease or cholesteryl ester storage disease (CESD). In additional embodiments, the disorder related to lipid storage or accumulation is coronary artery disease, atherosclerosis, type II diabetes, obesity, or nonalcoholic fatty liver disease (NAFLD).

In addition, described herein is the use of a polynucleotide, an rAAV, or an rAAV particle described herein in the preparation of a medicament for the treatment of dyslipidemia or hypercholesterolemia in a subject in need thereof.

The disclosure also provides for use of a polynucleotide, an rAAV, or an rAAV particle described herein in the preparation of a medicament for decreasing triglycerides, cholesterol, and/or fatty acids in a subject in need thereof.

For example, any of the disclosed medicaments are formulated for intravenous or intraperitoneal delivery. In some embodiments, the medicament is administered simultaneously, prior to, or after administration of an immunosuppressing agent, such as prednisone, prednisolone, rapamycin, methotrexate, myophenolate mofetil, tacrolimus, mycophenolate, or rituximab.

In another aspect, described herein is a composition comprising a polynucleotide, an rAAV, an rAAV particle, or a composition described herein for the treatment of LAL-D or a disorder related to lipid storage or accumulation. In some embodiments, the LAL-D is Wolman disease or cholesteryl ester storage disease (CESD). In additional embodiments, the disorder related to lipid storage or accumulation is coronary artery disease, atherosclerosis, type II diabetes, obesity, or nonalcoholic fatty liver disease (NAFLD).

In addition, described herein is a composition comprising a polynucleotide, an rAAV, an rAAV particle, or a composition described herein for the treatment of dyslipidemia or hypercholesterolemia in a subject in need thereof.

In a further aspect, described herein is a composition comprising a polynucleotide, an rAAV, an rAAV particle, or a composition described herein for decreasing triglycerides, cholesterol, and/or fatty acids in a subject in need thereof.

For example, any of the disclosed compositions are formulated for intravenous or intraperitoneal delivery. In some embodiments, the composition is administered simultaneously, prior to, or after administration of an immunosuppressing agent. In another embodiment, the composition further comprises an immunosuppressing agent. Exemplary, immunosuppressing agents include prednisone, prednisolone, rapamycin, methotrexate, mycophenolate mofetil, tacrolimus, or rituximab.

### BRIEF DESCRIPTION OF THE FIGURES

FIG. 1 provides a schematic to the rscAAVrh74.LP1.LIPA of the present disclosure.

FIG. 2 shows Lipase A enzyme activity after transfection of the Lipase A (LIPA, LAL) gene using various AAV promoter constructs. The promoter construct scAAV.miniCMV.LIPA was compared to single stranded (ss) or self-complementary (sc) AAV vectors using different constitutive promoters (Cbh, chick beta actin hybrid intron) or liver specific (ApoE or LP1) promoters. FIG. 2 shows that most promoters are superior to the miniCMV promoter in inducing LIPA activity. Errors are SD for n=3/grp.

FIG. 3 shows organ size after 4 month treatment with AAV vectors. Wild type (WT) and untreated Lipa^{-/-} mice were compared to Lipa^{-/-} treated with one of four AAVrh74 vectors intravenously (IV) at 2 months of age at a dose of 4×10¹³ vg/kg. All mice were analyzed at 6 months of age, after 4 months of treatment. FIG. 3A measures relative weight of the liver; FIG. 3B measures relative weight of the spleen; FIG. 3C measures relative weight of the intestine; and FIG. 3D measures relative weight of the lymph node. Errors are SD for n=3-7/grp. Stats are one-way ANOVA. *p<0.05, **p<0.01, ***p<0.001, ****p<0.0001.

FIG. 4 shows data comparing the relative weight of the liver and lymph node in Lipa^{-/-} mice treated with rscAAVrh74.LP1.LIPA LP1 and scAAV.miniCMV.LIPA at 2×10¹³ vg/kg IV dose. Errors are SD for n=1-7/grp. Stats are one-way ANOVA.

FIG. 5 shows triglyceride (TG) and cholesterol levels in the liver and spleen after AAV treatment in Lipa^{-/-} mice. Wild type (WT) and untreated Lipa^{-/-} mice were compared to Lipa^{-/-} treated with one of four AAVrh74 vectors intravenously (IV) at 2 months of age at a dose of 4×10¹³ vg/kg. All mice were analyzed at 6 months of age, after 4 months of treatment. FIG. 5A measures triglyceride levels in the liver; FIG. 5B measures triglyceride levels in the spleen; FIG. 5C measures cholesterol levels in the liver; and FIG. 5D measures cholesterol levels in the spleen. Errors are SD for n=3-7/grp. Stats are one-way ANOVA. *p<0.05, **p<0.01, ***p<0.001, ****p<0.0001.

FIG. 6 shows biodistribution of AAV vector genomes in tissues after IV dosing at 4×10¹³ vg/kg in Lipa^{-/-} mice. Vector genomes (vg) per nucleus were measured for four different AAV vectors after treatment of Lipa^{-/-} mice. Mice were treated intravenously (IV) at 2 months, with analysis at 6 months of age. The low transduction of both Cbh vectors in liver, suggesting immune clearance. Errors are SD for n=3/grp.

FIG. 7 shows human LIPA transgene expression, relative to endogenous mouse wild type Lipa gene expression, in tissues of Lipa^{-/-} mice. FIG. 7A shows transgene expression from various organs. FIG. 7B shows transgene expression from the liver only. Mice were treated intravenously (IV) at 2 months, with analysis at 6 months of age. LP1 gene expression in the liver is 216 times normal wild type mouse liver Lipa gene expression, while miniCMV is only 6 times normal. LP1 expression in other organs (e.g., muscle, lung, kidney, and heart) is quite low, consistent with the liver-specific nature of the LP1 promoter.

FIG. 8 shows LIPA enzyme activity in tissues after IV treatment of AAV vectors in Lipa^{-/-} mice. Wild type (WT) and untreated Lipa^{-/-} mice were compared to Lipa^{-/-} treated with one of four AAVrh74 vectors intravenously (IV) at 2 months of age at a dose of 4×10¹³ vg/kg. All mice were analyzed at 6 months of age, after 4 months of treatment. FIG. 8A measures enzyme activity in the liver; FIG. 8B measures enzyme activity in the serum; FIG. 8C measures enzyme activity in the spleen; FIG. 8D measures enzyme activity in the kidney;

FIG. 8E measures enzyme activity in the lungs; and FIG. 8F measures enzyme activity in the brain. Errors are SD for n=3-7/grp. Stats are one-way ANOVA. *p<0.05, **p<0.01, ***p<0.001, ****p<0.0001.

FIG. 9 provides an annotated sequence of sc.ptrs.LP1.LIPA KanR (5932 bp) in which the upper sequence is set out as SEQ ID NO: 5 and the lower sequence is SEQ ID NO: 6.

FIGS. 10A-D demonstrate improvement of hepatosplenomegaly by scAAVrh74.LP1.LIPA gene therapy. FIG. 10A provides the Injection scheme. FIG. 10B provides the liver (upper) and spleen (lower) appearance before and after treatments. FIG. 10C-D provide organ weights of liver (C) and spleen (D) at 6 months of age. Doses are all vg/kg body weight.

FIG. 11 provides images demonstrating reduction in lipid content after treatment with rscAAVrh74.LP1.LIPA gene therapy. Oil red O staining showed fat deposition in the liver before and after treatment. Hematoxylin (blue) is added as a counter stain. Doses are all vg/kg body weight.

FIG. 12 demonstrates rscAAV.LP1.LIPA gene therapy increased survival of treated Lipa^{-/-} mice. Untreated Lipa^{-/-} mice (red) do not survive beyond 305 days of age. Lipa^{-/-} mice treated at 2 months of age (60 days) all survive to beyond this time point. n=44 untreated Lipa^{-/-} mice, n=20; rscAAVrh74.LP1.LIPA-treated Lipa^{-/-} mice (dose is 1×10¹³ vg/kg).

FIGS. 13A-13H provide AAV biodistribution and LIPA gene expression in (A) liver, (B) spleen, (C) intestine, and (D) heart, and provide and LIPA gene expression in (E) liver, (F) spleen, (G) intestine, and (H) heart.

FIGS. 14A-14G provide liver-specific gene expression of rscAAVrh74.LP1.LIPA gene therapy in other organs: (A) lymph node, (B) lung, (C) kidney, (D) thymus, (E) brain or (F) gastrocnemius and (G) quadriceps.

FIGS. 15A-15J demonstrates LP1 enzyme activity is elevated in non-liver organs despite AAV-mediated LIPA gene expression being confined to the liver. Enzyme activity is shown in organs throughout the body: (A) liver (B) spleen, (C) intestine, (D) heart, (E) kidney, (F) lungs, (G) brain, (H) and (I) serum. FIG. 15J provides a Western blot showing expression of LIPA protein using an antibody against the protein for detection.

### DETAILED DESCRIPTION

While enzyme replacement protein therapy has shown clinical efficacy in WD and CESD patients and is approved by the FDA, such treatments require protein infusions every two weeks and give rise to only partial clinical correction (Burton et al. N Engl J Med 373: 1010-1020, 2015). In the phase 3 enzyme replacement clinical trial of Sebelipase Alfa (also called Kanuma), for example, elevations in serum aspartate aminotransferase (AST), alanine aminotransferase (ALT), total bilirubin, and low-density lipoprotein (LDL)-cholesterol, were, on average, reduced only by 50%, at best, with a 32% overall reduction in liver fat content and a 6.8% reduction in spleen volume (Burton et al. supra). As demonstrated herein, AAV delivery of the LIPA gene can have far greater impacts on these measures in a LAL-D mouse model.

The disclosure provides a recombinant (r) self-complementary (sc) AAV vector, rscAAVrh74.LP1.LIPA, for use in treating WD and CESD patients. The rhesus 74 (rh74) serotype of AAV, originally isolated from the spleen of a rhesus macaque has shown safety at high intravenous doses (2×10¹⁴ vg/kg) in clinical trials with pediatric patients. rAAVrh74 is similar to rAAV8, rAAV9, and rAAVrh10 in that it shows a high propensity to enter tissues after intravenous (IV) delivery to the blood, allowing for systemic multi-organ perfusion of the designed gene therapy using a single dose scheme (Zygmunt et al., Mol Ther Methods Clin Dev 15: 305-319, 2019). This dosing can last, in theory, at least in post-mitotic cells, for the lifetime of the animal (Chicoine et al., Mol Ther 22: 713-724, 2014; Martin et al., Am J Physiol Cell Physiol 296: C476-488, 2009). AAV is unique in its safety profile, as the viral genome, once transduced into its carrier cell, remains stably expressed as an episomal DNA and only very rarely ever integrates into the host genome (Grieger et al., Adv Biochem Eng Biotechnol 99: 119-145, 2005; Xiao et al. J Virol 72: 2224-2232, 1998). As with almost all AAV serotypes, liver and spleen are the most highly perfused organs when rAAVrh74 is delivered intravenously (Bish et al., Hum Gene Ther 19: 1359-1368, 2008). Liver typically receives logarithmically higher numbers of AAV DNA vector genomes (vg) than for other organs when adult animals are dosed, and this is true in multiple mammalian species, including humans, rhesus macaques, dogs, and rodents, including mice (Cunningham et al. Methods Mol Biol 1937: 213-219., 2019, Palaschak et al., Methods Mol Biol 1950: 333-360, 2019). Such features make AAV an ideal gene delivery method for treatment of genetic disorders such as LAL-D, where liver and spleen are the most affected organs (Aguisanda et al., supra; Burton et al. supra.).

rscAAV.LP1.LIPA disclosed herein is a new gene therapy vector that allows for liver-specific expression of the human lipase A (LIPA) gene in a self-complementary (sc) AAV genome configuration. LP1 contains the core liver-specific elements of the HCR (human alipoprotein E/C-gene locus control region) and human a1 anti-trypsin (hAAT) promoter. All of the data supporting this application will be using the rhesus 74 (rh74) serotype of AAV using, where the vector is introduced by intravenous administration at various doses. A separate technology utilizing rscAAV.miniCMV.LIPA, which allowed for expression of the human Lipase A gene in all tissues using a miniaturized Cytomegalovirus promoter (miniCMV) was previously disclosed in International Patent Publication No. WO 2022/164860. It was shown in a recent publication that this vector could both inhibit and reverse disease in a mouse model of Lysosomal Acid Lipase Deficiency (LALD) where the mouse Lipase A gene has been deleted (Lipa^{-/-}) (Lam et al, P., Mol. Ther. Methods Clin Devel. 26:413-426, 2022). This data showed that most, but not all, disease could be reversed if mice were dosed at 2 months of age and assayed at 6 months of age. In the absence of treatment, both liver and spleen in Lipa^{-/-} mice show profound changes indicative of LALD, with liver expanding to 5 times its normal size because of increased triglyceride and cholesterol deposition. The LIPA enzyme breaks down triglycerides to free fatty acids and cholesteryl esters to free cholesterol in the lysosome. Direct measure of triglyceride and cholesterol levels also showed a profound increase in these tissues without treatment that was largely reversed when gene therapy was introduced.

There are two important aspects of rscAAV.LP1.LIPA of the present disclosure that distinguish it from the rscAAV.miniCMV.LIPA disclosed in International Patent Publication No. WO 2022/164860 and that clearly make rscAAV.LP1.LIPA a superior product: 1. Use of the LP1 promoter allows for increased gene expression only in the liver, while miniCMV allows for expression of LIPA in all tissues, including in antigen presenting cells that may prime the immune system for immune rejection of the foreign protein. It is well documented that liver-specific expression of AAV can induce immune tolerance due to the upregulation of regulatory T cells (Tregs), which can reduce activation of both B and T cell responses to the transgenic protein. Thus, LP1 may be a safer gene therapy in LALD patients, particularly in patients where all enzyme expression has been lost (e.g., infants with Wolman disease). 2. rscAAV.LP1.LIPA not only allows for liver-specific gene expression, but it increases gene expression of LIPA in the liver 36-fold compared to the previous miniCMV technology. Because of this, much more LIPA protein is secreted from the liver, providing enzyme replacement therapy (ERT) for organs throughout the body. Because LIPA is a lysosomal enzyme, it can be secreted into the serum and then reinternalized in other organs and correctly targeted back to the lysosome in those organs. This is the concept of ERT protein therapy for lysosomal storage disorders. The present disclosure seeks to utilize this concept with gene therapy, where a single intravenous (IV) treatment will lead to permanent elevations in the lysosomal enzyme. This would be greatly preferred to ERT therapy because protein elevation would be chronic and consistently improved compared to ERT, which must be given every two weeks and where protein half-life is on the order of hours. LALD patients complain that the frequency required for ERT therapy, with perfusion every 1-2 weeks for their entire lifetime, is a major impediment to their quality of life.

This disclosure shows that the use of LP1 leads to 5-10 times the normal wild type amount of LIPA enzyme activity in non-liver organs, including spleen, lung, kidney, with even some enzyme elevation in the brain. This increased enzyme activity in non-liver tissues allows for superior long-term treatment in LALD patients. The present disclosure also show that the same biological effects can be achieved at a lower dose (2×10¹³ vg/kg), a dose where miniCMV begins to lose some potency. The improved technology of the present disclosure should allow for a dramatic clinical improvement over miniCMV in LALD patients, as miniCMV cannot elevate enzyme activity in non-liver tissues to nearly the same extent. It also may dramatically improve treatment of infants with Wolman disease, as secreted protein in the liver may provide ERT even as hepatocytes divide and AAV genomes are lost during liver growth. Lam et al. showed that miniCMV promoter, while therapeutic when dosed at postnatal day 2, lost potency over time. It is likely that the LP1 promoter will drive superior treatment in such cases due to increased ERT therapy. For these reasons, the rscAAVrh74.LP1.LIPA is a superior technology for use in patients with LALD compared to rscAAV.miniCMV.LIPA disclosed in International Patent Publication No. WO 2022/164860.

The disclosed AAV vector is optimized for therapeutic usefulness in LAL-D. The self-complementary (sc) technology allows for binding of the single-stranded viral DNA genome onto itself, thereby priming second strand DNA synthesis. This self-complimentary element both quickens and strengthens gene expression relative to constructs lacking the self-complimentary element. Use of the self-complimentary technology is important for effective treatment of LAL-D, as children with complete deficiency become severely ill within a week or two after birth. Thus, use of a single-stranded rAAV vector, which will take 3-4 week for maximal onset of gene expression, would not be ideal for preventing a disease with such an early and severe onset. Because only about 2.2 kB of DNA can be packaged into self-complementary AAV vectors, a little less than half what can normally be packaged in a single-stranded vector (4.7 kB), an LP1 promoter, which is small in size, was used to allow for all of the required DNA elements to be included along with the full-length human LIPA transgene.

### LIPA Mutations

The LIPA gene is located on human chromosome 10q23.2-23.3 and consists of 10 exons spread over approximately 38 kb. LIPA has 3 transcript variants: Variant 2 (NM_000235) lacks an internal segment in the 5′ UTR compared with variant 1 (NM_001127605). The two variants encode the same protein isoform in size of 399 amino acids (AAs), which has been experimentally validated by cDNA cloning (Baratta et al., World J Gastroenterol 25: 4172-4180). The annotated variant 3 (NM_001288979) lacks two consecutive exons in the 5′ region, which results in translation initiation at a downstream AUG and presumably a shorter protein isoform consists of 283 AAs. (Li and Zhang, Arterioscler Thromb Vasc Biol. 39(5): 850-856, 2019).

There are at least 59 known mutations in the LIPA gene. Examples of these mutations are provided in Table 1 below.

| DNA | Protein | Citation |
|---|---|---|
| | p.T16P | Morris et al. |
| | | Arterioscler Thromb |
| | | Vasc Biol. 2017 June; |
| | | 37(6): 1050-1057. |
| | p.T16W | Morris et al. |
| | | Arterioscler Thromb |
| | | Vasc Biol. 2017 June; |
| | | 37(6): 1050-1057. |
| | p.(Trp140*) | |
| | p.(Arg218*) | |
| | p.(Gly266*), | |
| c.67G > A | p.Gly23Arg | |
| c.129C > G | p, Tyr43* | Ries et al Hum |
| | | Mutat, 12 (1998), |
| | | pp. 44-51 |
| c.253C > A | p.Gln85Lys | |
| c.260G > T; | p.Gly87Val | |
| c.294C > G; | p.Asn98Lys | |
| c.229 + 3A > C | | Pisciotta et al. |
| | | Arterosclerosis |
| | | 265: 124-132, 2017 |
| c.386A > G | p.His129Arg | Consuelo-Sanchez |
| | | Ann. Hepatol. 18(4): |
| | | 646-650, 2019 |
| c.414dup; | p.Phe139llefs*7 | Mayatepek et al. |
| | | Hum |
| | | Mutat, 12 (1998) |
| | | pp. 44-51 |
| | p.(Thr288lle), | |
| | p.(Leu294Ser) | |
| c.822 + 37A > G | | |
| c.894G > A | p.Gln298Gln | Mutoni et al. Hum |
| | | Genet 95: 491-494, |
| | | 1995 |
| c.894G > A | p.delS275_Q298 | Consuelo-Sanchez |
| | | Ann. Hepatol. 18(4): |
| | | 646-650, 2019 |
| c.1024G > A; | p.Gly342Arg | Consuelo-Sanchez |
| | | Ann. Hepatol. 18(4): |
| | | 646-650, 2019 |
| | p.(Asp345Asn), | Pisciotta et al. |
| | | Arterosclerosis |
| | | 265: 124-132, 2017 |

### AAV Gene Therapy

The present disclosure provides for gene therapy vectors, e.g., rAAV vectors expressing the LIPA cDNA, and methods of treating Lysosomal Acid Lipase Deficiency (LAL-D), including Wolman disease and cholesteryl ester storage disease (CESD). The disclosed gene therapy vectors are useful for treating disorders related to lipid storage and accumulation such as coronary artery disease such as atherosclerosis, type II diabetes, obesity, and nonalcoholic fatty liver disease (NAFLD). In addition, the disclosed gene therapy vectors are useful for decreasing triglycerides, cholesterol, and/or fatty acids in a subject in need thereof, and for treating dyslipidemia or hypercholesterolemia in a subject in need thereof.

As used herein, the term “AAV” is a standard abbreviation for adeno-associated virus. Adeno-associated virus is a single-stranded DNA parvovirus that grows only in cells in which certain functions are provided by a co-infecting helper virus. There are currently at least thirteen serotypes of AAV that have been characterized. General information and reviews of AAV can be found in, for example, Carter, 1989, Handbook of Parvoviruses, Vol. 1, pp. 169-228, and Berns, 1990, Virology, pp. 1743-1764, Raven Press, (New York). However, it is fully expected that these same principles will be applicable to additional AAV serotypes since it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

An “AAV vector” as used herein refers to a vector comprising one or more polynucleotides of interest (or transgenes) that are flanked by AAV terminal repeat sequences (ITRs). Such AAV vectors can be replicated and packaged into infectious viral particles when present in a host cell that has been transfected with a vector encoding and expressing rep and cap gene products.

An “AAV virion” or “AAV viral particle” or “AAV vector particle” refers to a viral particle composed of at least one AAV capsid protein and an encapsidated polynucleotide AAV vector. If the particle comprises a heterologous polynucleotide (i.e., a polynucleotide other than a wild-type AAV genome such as a transgene to be delivered to a mammalian cell), it is typically referred to as an “AAV vector particle” or simply an “AAV vector”. Thus, production of AAV vector particle necessarily includes production of AAV vector, as such a vector is contained within an AAV vector particle.

Adeno-associated virus (AAV) is a replication-deficient parvovirus, the single-stranded DNA genome of which is about 4.7 kb in length including an inverted terminal repeat (ITRs). Exemplary ITR sequences may be 130 base pairs in length or 141 base pairs in length. Exemplary ITR sequences provided in the present disclosure include a 5′ ITR and a 3′ ITR, which contains a deletion of the terminal resolution site (referred to as “dTR”). Deletion of the terminal resolution site inhibits Rep protein nicking of the single stranded viral genome. The presence of the dTR in the 3′ ITR increases self-complementary binding of the viral genome to itself, which it may do because of its small (2.2 kB) size that allows for a double-stranded viral genome to be packaged within the viral capsid.

There are multiple serotypes of AAV. The nucleotide sequences of the genomes of the AAV serotypes are known. For example, the nucleotide sequence of the AAV serotype 2 (AAV2) genome is presented in Srivastava et al., J Virol, 45: 555-564 (1983) as corrected by Ruffing et al., J Gen Virol, 75: 3385-3392 (1994). As other examples, the complete genome of AAV-1 is provided in GenBank Accession No. NC_002077; the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively (see also U.S. Pat. Nos. 7,282,199 and 7,790,449 relating to AAV-8); the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). Cloning of the AAVrh.74 serotype is described in Rodino-Klapac, et al. Journal of Translational Medicine 5, 45 (2007). Cis-acting sequences directing viral DNA replication (rep), encapsidation/packaging and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (e.g., at AAV2 nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter, and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. The rep and cap proteins may be provided in trans. Another significant feature of AAV is that it is an extremely stable and hearty virus. It easily withstands the conditions used to inactivate adenovirus (56° C. to 65° C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. See, Clark et al., Hum Gene Ther, 8: 659-669 (1997); Kessler et al., Proc Nat. Acad Sc. USA, 93: 14082-14087 (1996); and Xiao et al., J Virol, 70: 8098-8108 (1996). See also, Chao et al., Mol Ther, 2:619-623 (2000) and Chao et al., Mol Ther, 4:217-222 (2001). Moreover, because muscle is highly vascularized, recombinant AAV transduction has resulted in the appearance of transgene products in the systemic circulation following intramuscular injection as described in Herzog et al., Proc Natl Acad Sci USA, 94: 5804-5809 (1997) and Murphy et al., Proc Natl Acad Sci USA, 94:13921-13926 (1997). Moreover, Lewis et al., J Virol, 76: 8769-8775 (2002) demonstrated that skeletal myofibers possess the necessary cellular factors for correct antibody glycosylation, folding, and secretion, indicating that muscle is capable of stable expression of secreted protein therapeutics.

Recombinant AAV genomes of the disclosure comprise nucleic acid molecule of the disclosure and one or more AAV ITRs flanking a nucleic acid molecule. AAV DNA in the rAAV genomes may be from any AAV serotype for which a recombinant virus can be derived including, but not limited to, AAV serotypes (e.g., AAV1, AAV2, AAV3, AAV4, AAV5, AAV6, AAV7, AAV8, AAV9, AAVRH10, AAVRH74, AAV11, AAV12, AAV13, or Anc80, AAV7m8 and their derivatives). Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). As noted in the Background section above, the nucleotide sequences of the genomes of various AAV serotypes are known in the art.

The provided recombinant AAV (i.e., infectious encapsidated rAAV particles) comprise a rAAV genome. The term “rAAV genome” refers to a polynucleotide sequence that is derived from a native AAV genome that has been modified. In some embodiments, the rAAV genome has been modified to remove the native cap and rep genes. In some embodiments, the rAAV genome comprises the endogenous 5′ and 3′ inverted terminal repeats (ITRs). In some embodiments, the rAAV genome comprises ITRs from an AAV serotype that is different from the AAV serotype from which the AAV genome was derived. In some embodiments, the rAAV genome comprises a transgene of interest flanked on the 5′ and 3′ ends by inverted terminal repeat (ITR). In some embodiments, the rAAV genome comprises a “gene cassette.” In exemplary embodiments, the genomes of both rAAV lack AAV rep and cap DNA, that is, there is no AAV rep or cap DNA between the ITRs of the genomes.

The rAAV genomes provided herein, in some embodiments, comprise one or more AAV ITRs flanking the transgene polynucleotide sequence. The transgene polynucleotide sequence is operatively linked to transcriptional control elements (including, but not limited to, promoters, enhancers, and/or polyadenylation signal sequences) that are functional in target cells to form a gene cassette. Examples of promoters are the LP1 promoter having the nucleotide sequence of SEQ ID NO: 3. Additional promoters are contemplated herein including, but not limited to the hAAT promoter, ApoE promoter, HCR control region, FRE1 promoter, albumin (ALB) promoter, transthyretin (TTR) promoter, hepatitis B virus (HBV) promoter, α1-antitrypsin promoter alone or linked to the albumin or hepatitis B enhancers, TBG, HLP, chicken β actin promoter (CBA), the P546 promoter the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter.

Additionally provided herein are a LP1 promoter sequence, and promoter sequences at least: 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of the LP1 (SEQ ID NO: 3) sequence which exhibit transcription promoting activity.

Other examples of transcription control elements are tissue specific control elements, for example, promoters that allow expression specifically within neurons or specifically within astrocytes. Examples include neuron specific enolase and glial fibrillary acidic protein promoters. Inducible promoters are also contemplated. Non-limiting examples of inducible promoters include, but are not limited to a metallothionine promoter, a glucocorticoid promoter, a progesterone promoter, and a tetracycline-regulated promoter. The gene cassette may also include intron sequences to facilitate processing of a transgene RNA transcript when expressed in mammalian cells. One example of such an intron is the SV40 intron.

rAAV genomes provided herein comprises a polynucleotide (SEQ ID NO: 1) encoding LIPA protein. In some embodiments, the rAAV genomes provided herein comprises a polynucleotide that encodes a polypeptide comprising an amino acid sequence that is at least: 90%, 91%, 92%, 93%, 94%, 95%, 96%, 97%, 98%, or 99% identical to the amino acid sequence encoded by the LIPA cDNA (SEQ ID NO 1).

rAAV genomes provided herein comprises a nucleotides 1853-4094 of SEQ ID NO: 4. In some embodiments, the rAAV genomes provided herein comprises a polynucleotide that at least: 90%, 91%, 92%, 93%, 94%, 95%, 96%, 97%, 98%, or 99% identical to the nucleotide sequences of nucleotides 1853-4094 of SEQ ID NO: 4.

rAAV genomes provided herein, in some embodiments, a polynucleotide sequence that encodes an LAL protein and that hybridizes under stringent conditions to the polynucleotide sequence set forth in SEQ ID NO: 1 or the complement thereof.

DNA plasmids of the disclosure comprise rAAV genomes of the disclosure. The DNA plasmids are transferred to cells permissible for infection with a helper virus of AAV (e.g., adenovirus, E1-deleted adenovirus, or herpesvirus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs, including, but not limited to, AAV serotypes AAV-9, AAV-1, AAV-2, AAV-3, AAV-4, AAV-5, AAV-6, AAV-7, AAVrh.74, AAV-8, AAV-10, AAV-11, AAV-12, and AAV-13. Production of pseudotyped rAAV is disclosed in, for example, WO 01/83692 which is incorporated by reference herein in its entirety.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., Mol. Cell. Biol., 7:349 (1988); Samulski et al., J. Virol., 63:3822-3828 (1989); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. Vaccine 13:1244-1250 (1995); Paul et al. Human Gene Therapy 4:609-615 (1993); Clark et al. Gene Therapy 3:1124-1132 (1996); U.S. Pat. Nos. 5,786,211; 5,871,982; and 6,258,595. The foregoing documents are hereby incorporated by reference in their entirety herein, with particular emphasis on those sections of the documents relating to rAAV production.

The disclosure thus provides packaging cells that produce infectious rAAV. In one embodiment packaging cells may be stably transformed cancer cells such as HeLa cells, 293 cells, and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells), and FRhL-2 cells (rhesus fetal lung cells).

The rAAV may be purified by methods standard in the art such as by column chromatography or cesium chloride gradients. Methods for purifying rAAV vectors from helper virus are known in the art and include methods disclosed in, for example, Clark et al., Hum. Gene Ther., 10(6): 1031-1039 (1999); Schenpp and Clark, Methods Mol. Med., 69 427-443 (2002); U.S. Pat. No. 6,566,118; and WO 98/09657.

Compositions provided herein comprise rAAV and a pharmaceutically acceptable excipient or excipients. Acceptable excipients are nontoxic to recipients and are preferably inert at the dosages and concentrations employed, and include, but are not limited to, buffers such as phosphate (e.g., phosphate-buffered saline (PBS)), citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, copolymers such as poloxamer 188, pluronics (e.g., Pluronic F68) or polyethylene glycol (PEG).

Dosages are expressed in units of vg/kg. Dosages contemplated herein include about 1×10¹¹ vg/kg, about 1×10¹² vg/kg, about 5×10¹² vg/kg, about 1×10¹³ vg/kg, about 1.1×10¹³ vg/kg, about 1.2×10¹³ vg/kg, about 1.3×10¹³ vg/kg, about 1.5×10¹³ vg/kg, about 2×10¹³ vg/kg, about 2.5×10¹³ vg/kg, about 3×10¹³ vg/kg, about 3.5×10¹³ vg/kg, about 4×10¹³ vg/kg, about 4.5×10¹³ vg/kg, about 5×10¹³ vg/kg, about 6×10¹³ vg/kg, about 7×10¹³ vg/kg, to about 8×10¹³ vg/kg.

Dosages contemplated herein include about 1×10¹¹ vg/kg, about 1×10¹² vg/kg, about 5×10¹² vg/kg, about 1×10¹³ vg/kg, about 1.1×10¹³ vg/kg, about 1.2×10¹³ vg/kg, about 1.3×10¹³ vg/kg, about 1.5×10¹³ vg/kg, about 2×10¹³ vg/kg, about 2.5×10¹³ vg/kg, about 3×10¹³ vg/kg, about 3.5×10¹³ vg/kg, about 4×10¹³ vg/kg, about 4.5×10¹³ vg/kg, about 5×10¹³ vg/kg, about 6×10¹³ vg/kg, about 7×10¹³ vg/kg, to about 8×10¹³ vg/kg. Dosages of about 1×10¹¹ vg/kg to about 1×10¹¹ vg/kg, about 1×10¹¹ vg/kg to about 1×10¹³ vg/kg, about 1×10¹² vg/kg to about 1×10¹⁴ vg/kg, about 1×10¹³ vg/kg to about 2×10¹⁴ vg/kg, about 1×10¹³ vg/kg to about 1×10¹⁴ vg/kg, about 2×10¹³ to about 4×10¹³, about 1.5×10¹³ to about 4.5×10¹³ and about 6×10¹³ vg/kg to about 1.0×10¹⁴ vg/kg are also contemplated. One dose exemplified herein is 2×10¹³ vg/kg administered via intravenous or intraperitoneal delivery.

Methods of transducing a target cell with rAAV, in vivo or in vitro, are contemplated by the disclosure. The in vivo methods comprise the step of administering an effective dose, or effective multiple doses, of a composition comprising a rAAV of the disclosure to an animal (including a human being) in need thereof. If the dose is administered prior to development of a disorder/disease, the administration is prophylactic. If the dose is administered after the development of a disorder/disease, the administration is therapeutic. In embodiments of the disclosure, an effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. Example of a LAL-D contemplated for prevention or treatment with methods of the disclosure is Wolman disease and cholesteryl ester storage disease (CESD) or a disorder related to lipid storage or accumulation such as coronary artery disease, atherosclerosis, type II diabetes, obesity, nonalcoholic fatty liver disease (NAFLD), dyslipidemia, or hypercholesterolemia.

Combination therapies are also contemplated by the disclosure. Combination as used herein includes both simultaneous treatment and sequential treatments. Combinations of methods of the disclosure with standard medical treatments are specifically contemplated, as are combinations with novel therapies. In some embodiments, the combination therapy comprises administering an immunosuppressing agent in combination with the gene therapy disclosed herein.

Administration of an effective dose of the compositions may be by routes standard in the art including, but not limited to, intramuscular, parenteral, intravenous, intraarterial, intraperitoneal, oral, buccal, nasal, pulmonary, intracranial, intraosseous, intraocular, rectal, or vaginal. Route(s) of administration and serotype(s) of AAV components of the rAAV (in particular, the AAV ITRs and capsid protein) of the disclosure may be chosen and/or matched by those skilled in the art taking into account the infection and/or disease state being treated and the target cells/tissue(s) that are to express the wild type LAL protein.

The disclosure provides for local administration and systemic administration of an effective dose of rAAV and compositions of the disclosure. For example, systemic administration is administration into the circulatory system so that the entire body is affected. Systemic administration includes enteral administration such as absorption through the gastrointestinal tract and parenteral administration through injection, infusion, or implantation.

Transduction of cells with rAAV of the disclosure results in sustained expression of the LAL protein. The present disclosure thus provides methods of administering/delivering rAAV which express LAL protein to an animal, preferably a human being. These methods include transducing cells with one or more rAAV of the present disclosure.

The term “transduction” is used to refer to the administration/delivery of the coding region of the LIPA gene to a recipient cell either in vivo or in vitro, via a replication-deficient rAAV of the disclosure resulting in expression of LAL the recipient cell.

### Immunosuppressing Agents

The immunosuppressing agent may be administered before or after the onset of an immune response to the rAAV in the subject after administration of the gene therapy. In addition, the immunosuppressing agent may be administered simultaneously with the gene therapy or the protein replacement therapy. The immune response in a subject includes an adverse immune response or an inflammatory response following or caused by the administration of rAAV to the subject. The immune response may be the production of antibodies in the subject in response to the administered rAAV.

Exemplary immunosuppressing agents include glucocorticosteroids, janus kinase inhibitors, calcineurin inhibitors, mTOR inhibitors, cyctostatic agents such as purine analogs, methotrexate and cyclophosphamide, inosine monophosphate dehydrogenase (IMDH) inhibitors, biologics such as monoclonal antibodies or fusion proteins and polypeptides, and di peptide boronic acid molecules, such as Bortezomib.

The immunosuppressing agent may be an anti-inflammatory steroid, which is a steroid that decreases inflammation and suppresses or modulates the immune system of the subject. Exemplary anti-inflammatory steroid are glucocorticoids such as prednisolone, betamethasone, dexamethasone, methotrexate, hydrocortisone, methylprednisolone, deflazacort, budesonide or prednisone.

Janus kinase inhibitors are inhibitors of the JAK/STAT signaling pathway by targeting one or more of the Janus kinase family of enzymes. Exemplary janus kinase inhibitors include tofacitinib, baricitinib, upadacitinib, peficitinib, and oclacitinib.

Calcineurin inhibitors bind to cyclophilin and inhibits the activity of calcineurin. Exemplary calcineurine inhibitors includes cyclosporine, tacrolimus and picecrolimus.

mTOR inhibitors reduce or inhibit the serine/threonine-specific protein kinase mTOR. Exemplary mTOR inhibitors include rapamycin (also known as sirolimus), everolimus, and temsirolimus.

The immunosuppressing agents include immune suppressing macrolides. The term “immune suppressing macrolides” refer to macrolide agents that suppresses or modulates the immune system of the subject. A macrolide is a class of agents that comprise a large macrocyclic lactone ring to which one or more deoxy sugars, such as cladinose or desoamine, are attached. The lactone rings are usually 14-, 15-, or 16-membered. Macrolides belong to the polyketide class of agents and may be natural products. Examples of immunosuppressing macrolides include tacrolimus, pimecrolimus, and rapamycin (also known as sirolimus).

Purine analogs block nucleotide synthesis and include IMDH inhibitors. Exemplary purine analogs include azathioprine, mycophenolate such as mycophenolate acid or mycophenolate mofetil and leflunomide.

Exemplary immunosuppressing biologics include abatacept, adalimumab, anakinra, certolizumab, etanercept, golimumab, infliximab, ixekizumab, natalizumab, rituximab, secukinumab, tocilizumab, ustekinenumab, vedolizumab, basiliximab, belatacep, and daclizumab.

In particular, the immunosuppressing agent is an anti-CD20 antibody. The term anti-CD20 specific antibody refers to an antibody that specifically binds to or inhibits or reduces the expression or activity of CD20. Exemplary anti-CD20 antibodies include rituximab, ocrelizumab, or ofatumumab.

Additional examples of immunosuppressing antibodies include anti-CD25 antibodies (or anti-IL2 antibodies or anti-TAC antibodies) such as basiliximab and daclizumab, and anti-CD3 antibodies such as muromonab-CD3, otelixizumab, teplizumab, and visilizumab, anti-CD52 antibodies such as alemtuzumab.

The following EXAMPLES are provided by way of illustration and not limitation. Described numerical ranges are inclusive of each integer value within each range and inclusive of the lowest and highest stated integer.

### EXAMPLES

### Example 1

### Gene Therapy Constructs Encoding LIPA Under the Control of an LP1 Promoter

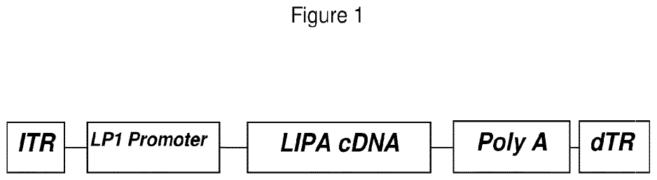

AAV genome constructs encoding LIPA were generated as set forth in FIG. 1, which depicts the AAVrh74 vector design with the full-length transcript of LIPA cDNA under the control of a LP1 promoter (SEQ ID NO: 3).

A human GFP cDNA clone was obtained from Origene, Rockville, MD. The LIPA cDNA alone was further subcloned into a self-complementary AAVrh74 genome under the control of an LP1 promoter. The plasmid construct also included an intron such as the simian virus 40 (SV40) chimeric intron and a polyadenylation signal (PolyA). The constructs were packaged into either AAVrh74 genome.

The LIPA cDNA expression cassette had a Kanamycin resistance gene, and an optimized Kozak sequence, which allows for more robust transcription. rAAV vectors were produced by a modified cross-packaging approach whereby the AAVrh74 vector genome can be packaged into multiple AAV capsid serotypes [Rabinowitz et al., J Virol. 76 (2):791-801 (2002)]. Production was accomplished using a standard three plasmid DNA/CaPO₄ precipitation method using HEK293 cells. HEK293 cells were maintained in DMEM supplemented with 10% fetal bovine serum (FBS) and penicillin and streptomycin. The production plasmids were: (i) plasmids encoding the therapeutic proteins, (ii) rep2-capX modified AAV helper plasmids encoding cap serotype AAVrh74 isolate, and (iii) an adenovirus type 5 helper plasmid (pAdhelper) expressing adenovirus E2A, E4 ORF6, and VA I/II RNA genes. A quantitative PCR-based titration method was used to determine an encapsidated vector genome (vg) titer utilizing a Prism 7500 Taqman detector system (PE Applied Biosystems). [Clark et al., Hum Gene Ther. 10 (6): 1031-1039 (1999)]. A final titer (vg ml⁻¹) was determined by quantitative reverse transcriptase PCR using the specific primers and probes utilizing a Prism 7500 Real-time detector system (PE Applied Biosystems, Grand Island, NY, USA). Aliquoted viruses were kept at −80° C.

All plasmids used to make AAV genomes to be packaged also contain a Kanamycin resistance gene (KanR) outside of the ITR sequences used for packaging of the genome. This allows for the DNA encoding the AAV genome to be transformed into bacteria to produce large amounts of DNA in the presence of Kanamycin, which will kill all non-transformed bacteria. KanR is not packaged into the AAV capsid in the AAV genome used to treat patients, but its presence allows for DNA production in bacteria.

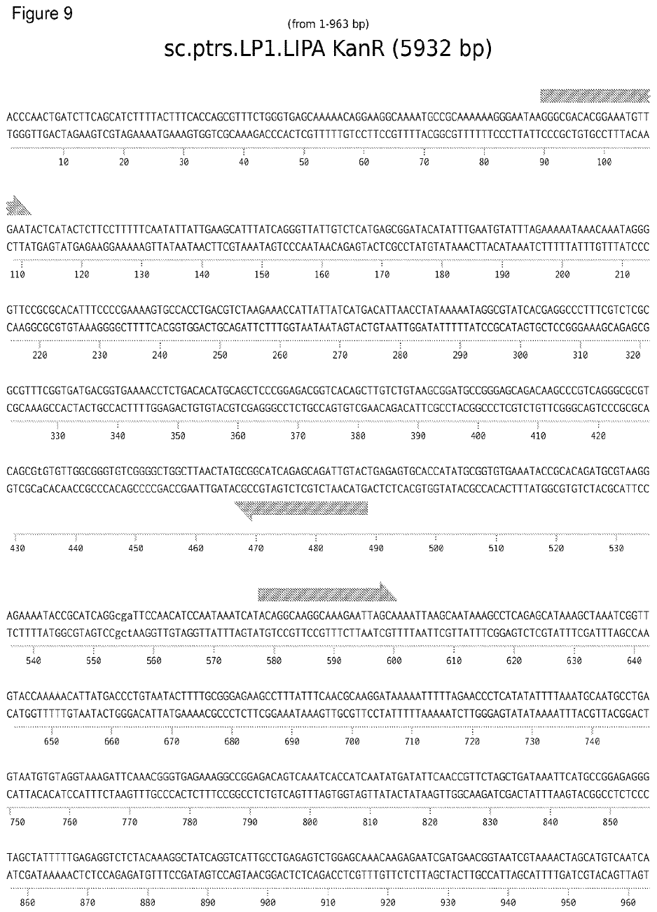

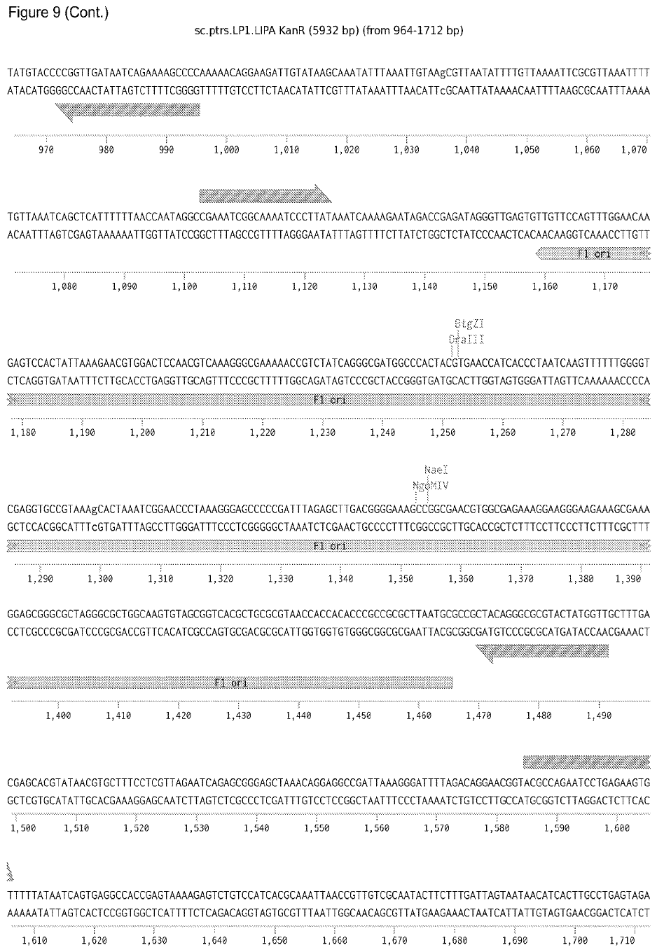

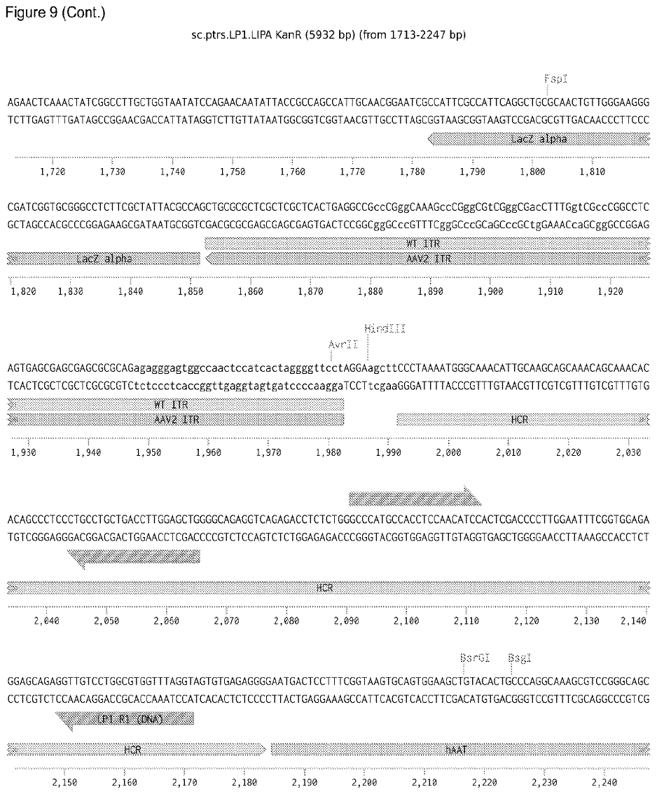

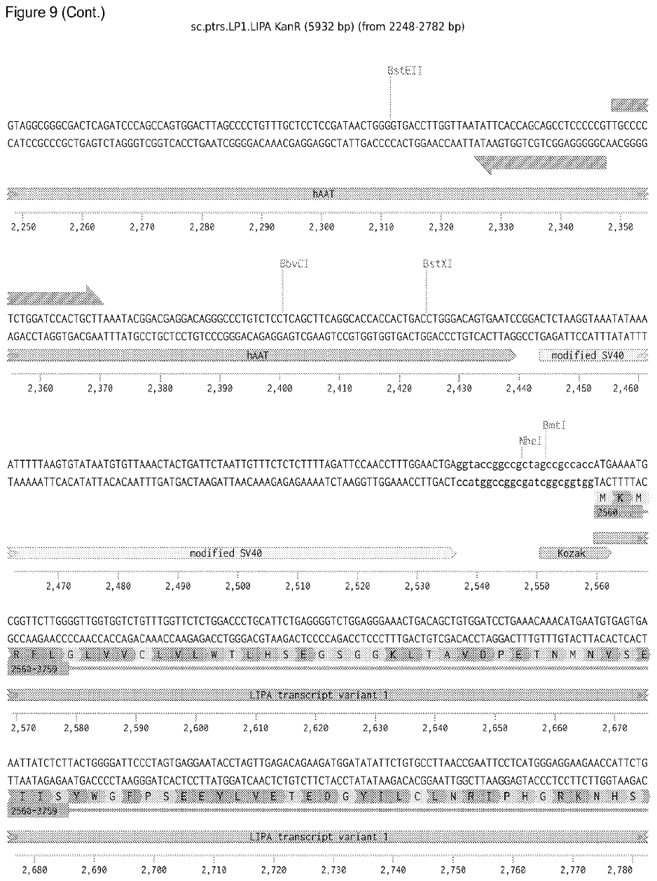

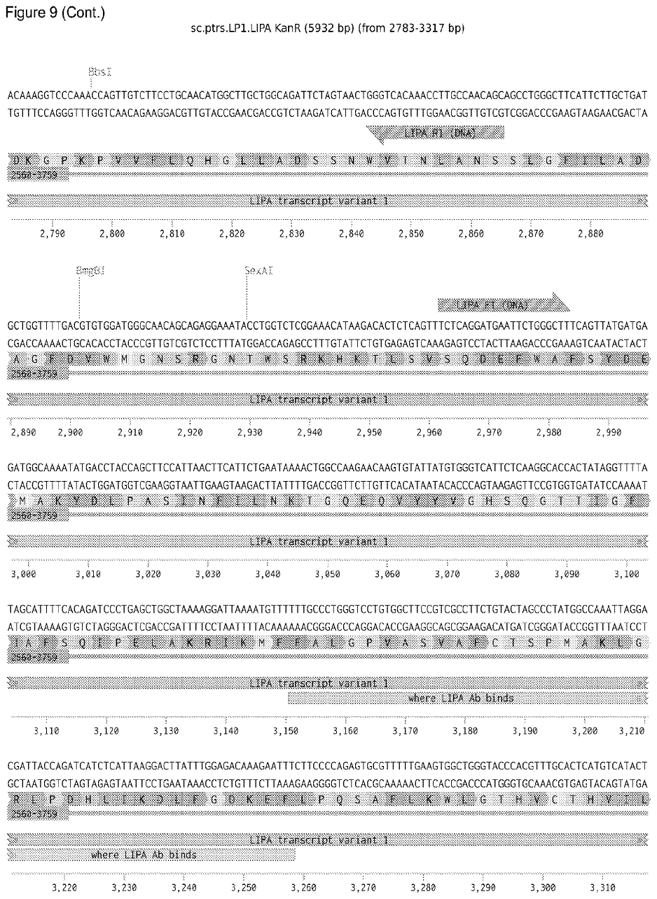

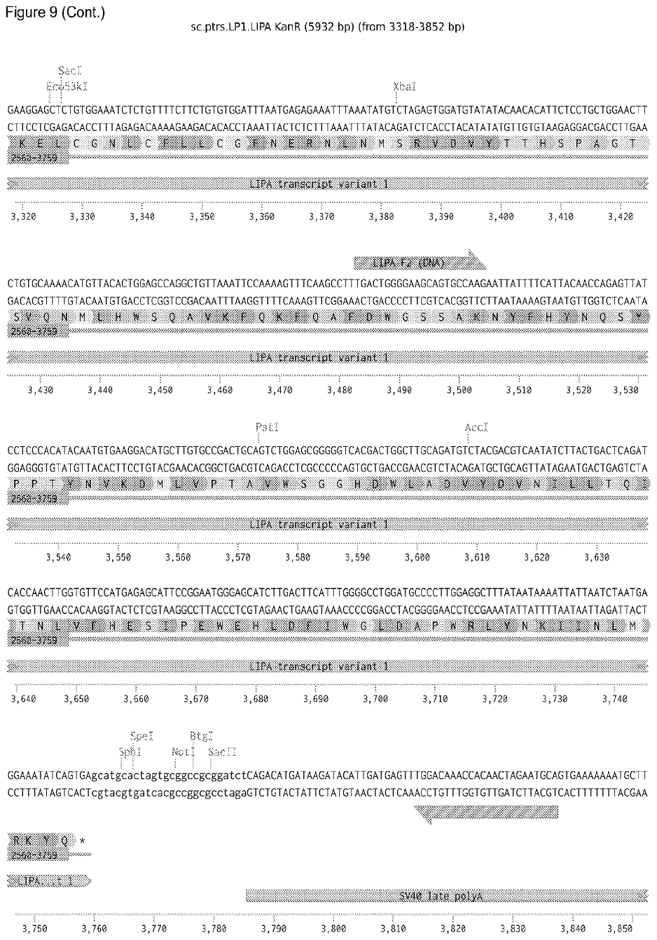

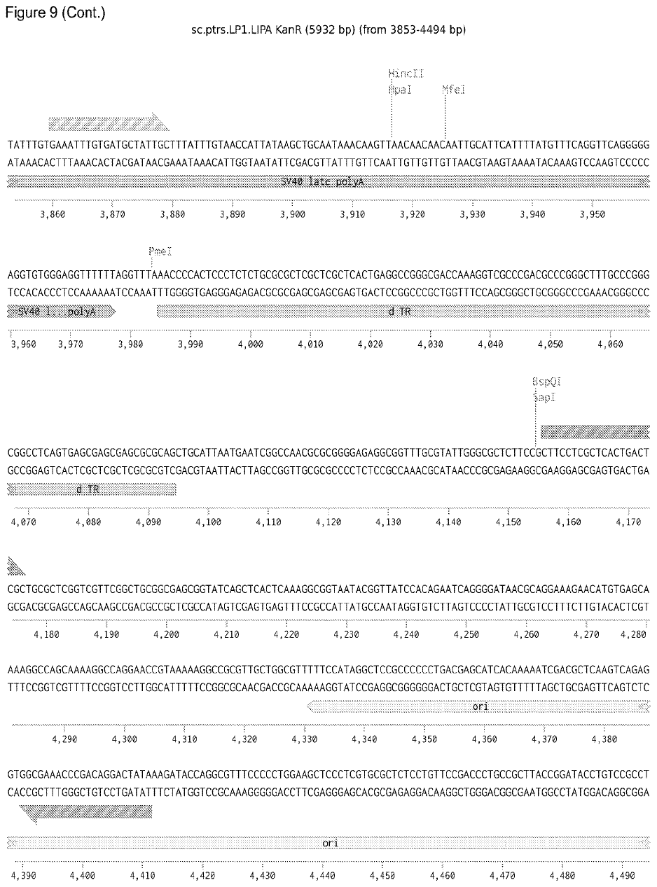

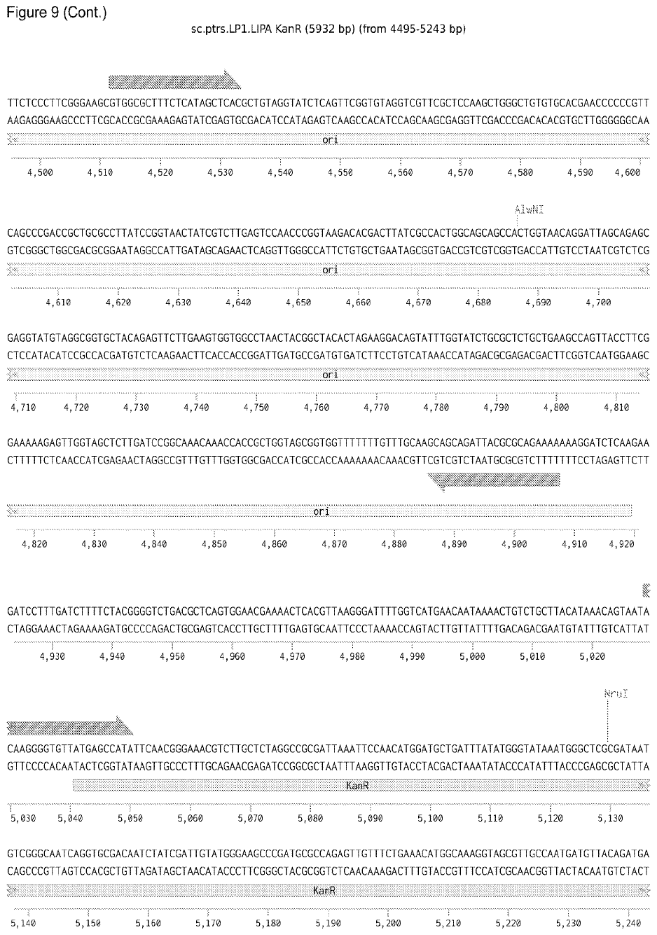

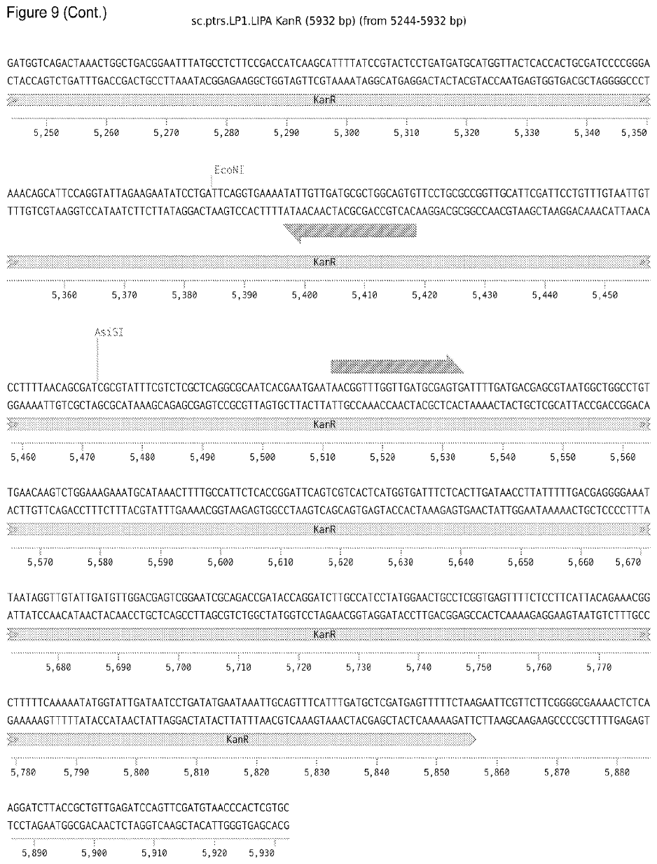

The map for plasmid r(sc) AAVrh74.LP1.LIPA is set out in FIG. 9 and the sequence of the entire plasmid is provided in SEQ ID NO: 4. The vector scAAVrh74.LP1.LIPA comprises the nucleotide sequence within and inclusive of the ITR's of SEQ ID NO: 4. The rAAV vector comprises the 5′ AAV2 ITR, LP1 promoter, the coding sequence for the LIPA gene, SV40 late polyA, and 3′ AAV2 ITR. The plasmid set forth in SEQ ID NO: 4 further comprises kanamycin resistance with pUC origin of replication.

Table 2 shows the molecular features of the plasmid (SEQ ID NO: 4), in which range refers to the nucleotides in SEQ ID NO: 4 and [symbol] indicates the kanamycin gene is in the forward orientation.

| Name | Range | Strand | Length |
|---|---|---|---|
| F1 ori | 1158-1465 | [symbol] | 308 |
| LacZ alpha | 1783-1851 | [symbol] | 69 |
| Wt 5′ ITR | 1853-1982 | [symbol] | 130 |
| LP1 (HCR + hAAT) | 1992-2439 | [symbol] | 448 |
| HCR | 1992-2183 | [symbol] | 192 |
| hAAT | 2184-2439 | [symbol] | 256 |
| SV40 intron | 2444-2536 | [symbol] | 93 |
| LIPA transcript | 2560-3759 | [symbol] | 1200 |
| variant 1 | | | |
| SV40 late polyA | 3786-3977 | [symbol] | 192 |
| Wt 3′ d TR | 3985-4094 | [symbol] | 110 |
| ori | 4331-4919 | [symbol] | 589 |
| KanR | 5041-5856 | [symbol] | 816 |

### Example 2

### Generation of LIPA-Deficient Mice

The following study tested AAV-based LIPA (human) gene replacement therapy in pure-bred Lipa-deficient (Lipa^{-/-}) mice. Lipa^{-/-} mice, like WD and CESD patients, develop severe liver dysfunction and damage as the result of the massive loading of cholesterol esters and triglycerides into this organ (Du et al. Hum Mol Genet 7: 1347-1354, 1998; Du et al. J Lipid Res 42: 489-500, 2001). For example, the mice develop hepatosplenomegaly, elevated serum aspartate aminotransferase (AST), alanine aminotransferase (ALT), and elevated liver and spleen cholesterol and triglycerides. Disease is evident within 3 weeks of birth, and Lipa^{-/-} mice have profound disease by 4 months, showing a 3- to 6-fold increase in the size of the liver and the spleen. Mice succumb to disease several months thereafter, beginning at 6 months of age.

Mice: The lal^{-/-} mice were first generated by Du et al in 1998. The mouse model has been widely used to study the role of Lal in multiple organ systems. The lal^{-/-} mice on a mixed genetic background of 129Sv and CF-1 survive into adulthood, and are fertile, but die at ages of 7 to 8 months. The lal^{-/-} mice show massive accumulation of TGs and CEs in liver, spleen, and small intestine. These mice resemble hepatosplenomegaly, the major clinical manifestation of WD and CESD in human. The lal^{-/-} mice provide a model to study human WD and CESD, but more importantly, serve as a powerful tool to investigate the systemic impacts of lysosomal lipolysis on metabolic and immune homeostasis.

### Example 3

### Comparison of Promoter Constructs for Inducing LIPA Enzyme Activity

Different promoter constructs, i.e., with LP1 promoter, Cbh (chick beta actin hybrid intron) promoter, and miniCMV promoter, were made in the same AAV vector to compare promoter function in increasing LIPA enzyme activity in HepG2 human liver cells. The HepG2 cells were transduced with the various AAV vectors.

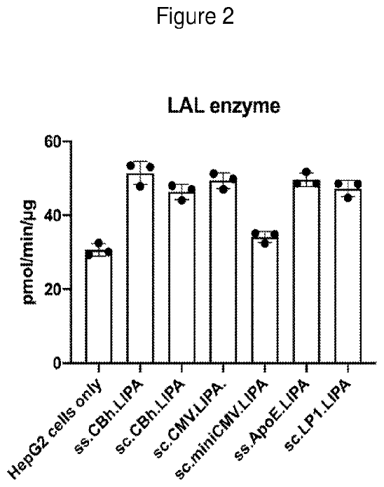

FIG. 2 illustrates Lipase A enzyme activity after transfection of the Lipase A (LIPA, LAL) gene using various AAV promoter constructs. The promoter construct scAAV.miniCMV.LIPA was compared to single stranded (ss) or self-complementary (sc) AAV vectors using different constitutive (Cbh, chick beta actin hybrid intron) or liver specific (ApoE or LP1) promoters. As shown in FIG. 2, most promoters are superior to the miniCMV promoter in inducing LIPA activity. In particular, the constitutive Cbh promoter and the liver-specific LP1 promoter were both superior to the miniCMV promoter in inducing LIPA enzyme (LAL) activity (FIG. 2).

### Example 4

### Comparison of Intravenous (IV) Treatments by Various Constructs

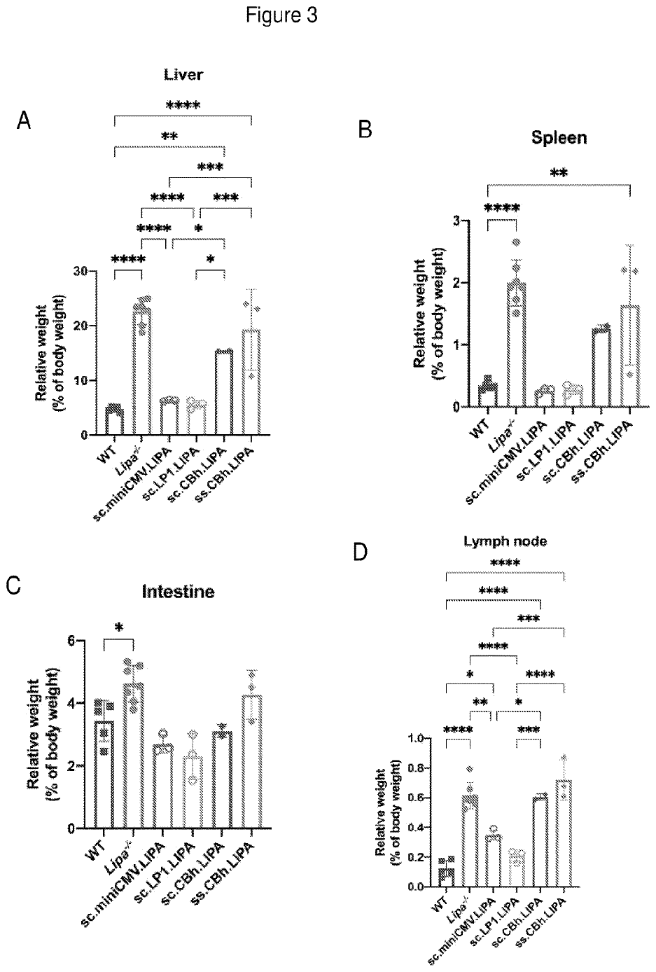

FIG. 3 shows organ size 4 months post-treatment with AAV vectors. Wild type (WT) and untreated Lipa^{-/-} mice were compared to Lipa^{-/-} treated with one of four AAVrh74 vectors intravenously (IV) at 2 months of age at a dose of 4×10¹³ vg/kg. All mice were analyzed at 6 months of age, after 4 months of treatment.

Intravenous (IV) treatment of rssAAVrh74.Cbh.LIPA, rscAAVrh74.Cbh.LIPA, and rscAAVrh74.LP1.LIPA was compared to that of rscAAVrh74.miniCMV.LIPA in Lipa^{-/-} mice. Mice were dosed at 2 months of age, a time when disease is already present and clearly significant, and assayed for at 6 months of age, 4 months after treatment, a point by which LALD disease is quite severe (most mice begin to perish from the disease between 6 and 7 months of age).

The first phenotype investigated was hepatosplenomegaly (FIG. 3). Liver and spleen size increased dramatically in untreated Lipa^{-/-} mice relative to wild type, such that the liver is about 4.8 times normal size by 6 months of age. AAV vectors having the LP1 promoter were as good as AAV having the miniCMV promoters at reducing liver and spleen size, while both scAAV.Cbh.LIPA and ssAAV.Cbh.LIPA had less therapeutic impact. Similarly, there was a greater reduction in the size of the intestine and lymph node in mice treated with scAAV.LP1.LIPA than when treated with scAAV.miniCMV.LIPA, while again vectors comprising Cbh, such as scAAV.Cbh.LIPA and ssAAV.Cbh.LIPA, showed less of an effect. Treatment with the scAAV.LP1.LIPA reduced the increase in lymph node size by 2-fold relative to scAAV.miniCMV.LIPA.

Mice were also treated with a lower dose of scAAV.LP1.LIPA and scAAV.miniCMV.LIPA (2×10¹³ vg/kg IV dose).

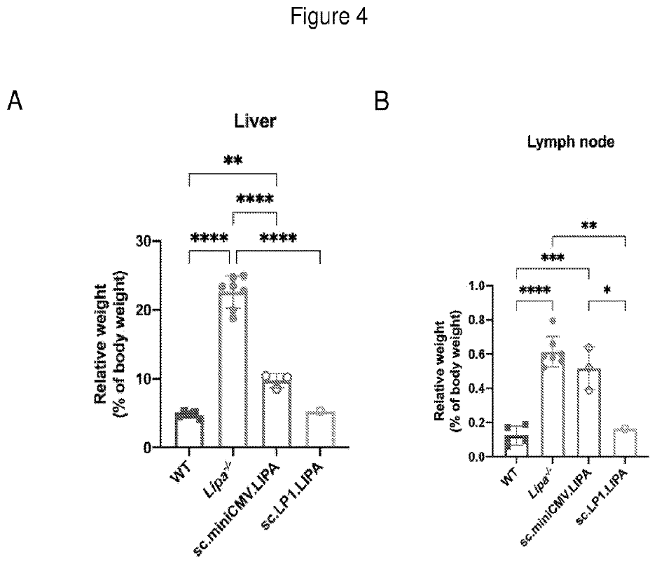

As shown in FIG. 4A, at the lower dose of 2×10¹³ vg/kg, the effects of rscAAV.miniCMV.LIPA on liver and especially on lymph node begin to decrease. However, the scAAV.LP1.LIPA treatment at the low dose showed equivalent effects as the higher 4×10¹³ vg/kg dose (FIG. 4). This data suggests, that inclusion of the LP1 may have biological effects that can be accomplished at a lower dose than is the case for rscAAV.miniCMV.LIPA.

### Example 5

### Comparison of Triglyceride and Cholesterol Levels of Various Constructs

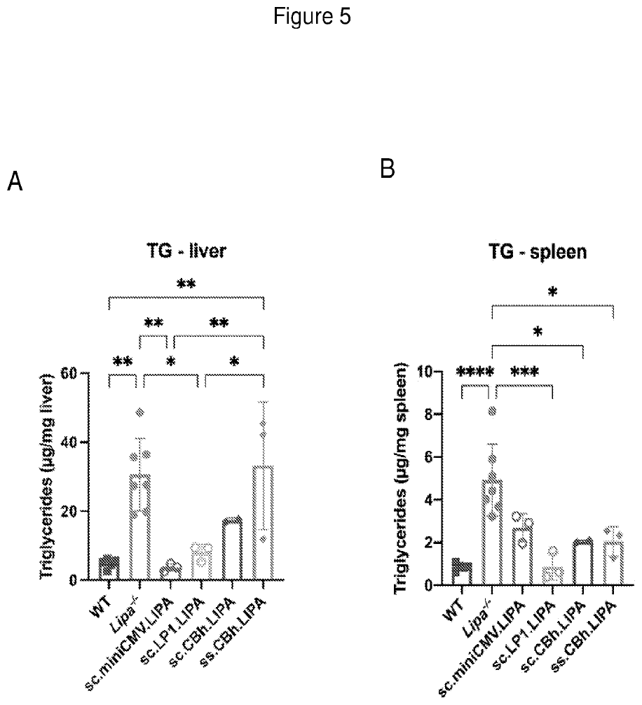

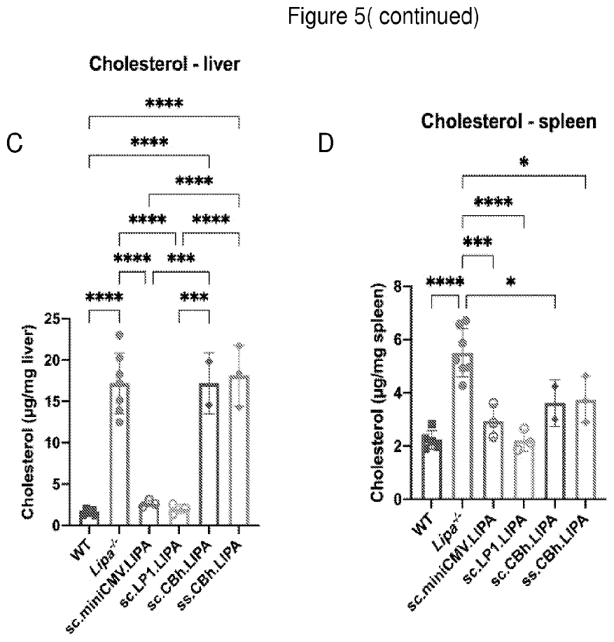

Next, the effect of IV administration of the four AAV vectors, e.g., scAAV.miniCMV.LIPA, scAAV.LP1.LIPA, scAAV.Cbh.LIPA, ssAAV.Cbh.LIPA, at a dose of 4×10¹³ vg/kg was investigated (FIG. 5). Wild type (WT) and untreated Lipa^{-/-} mice were compared to Lipa/treated with one of four AAVrh74 vectors intravenously (IV) at 2 months of age at a dose of 4×10¹³ vg/kg. All mice were analyzed at 6 months of age, after 4 months of treatment. All mice were analyzed at 6 months of age, after 4 months of treatment. Again, the inclusion of the LP1 promoter was either as good or better than the inclusion of the miniCMV promoter for lowering triglyceride and cholesterol levels in the liver and spleen. Similarly, both Cbh vectors (scAAV.Cbh.LIPA and ssAAV.Cbh.LIPA) were far poorer at lowering these levels, with effects sometimes (e.g., liver cholesterol) being negligible.

### Example 6

### Comparison of AAV Vector Genome Biodistribution of Various Constructs

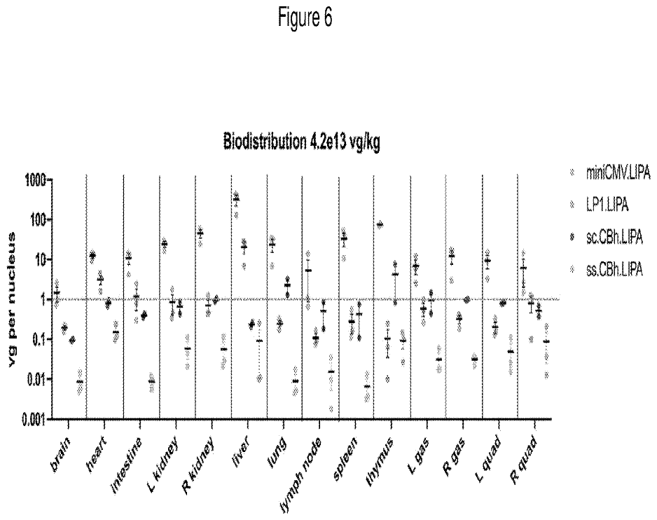

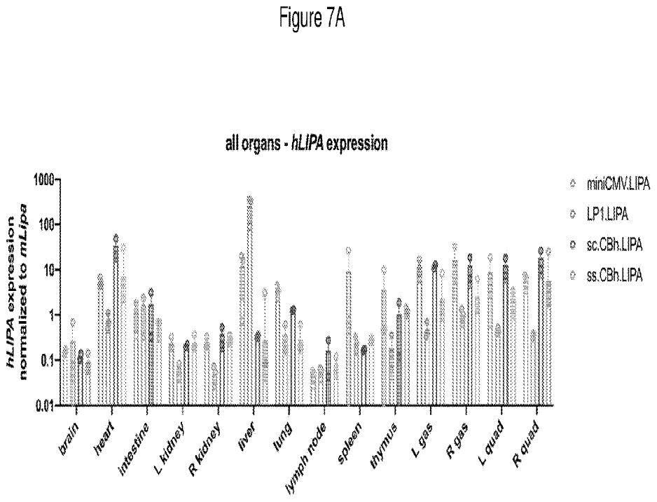

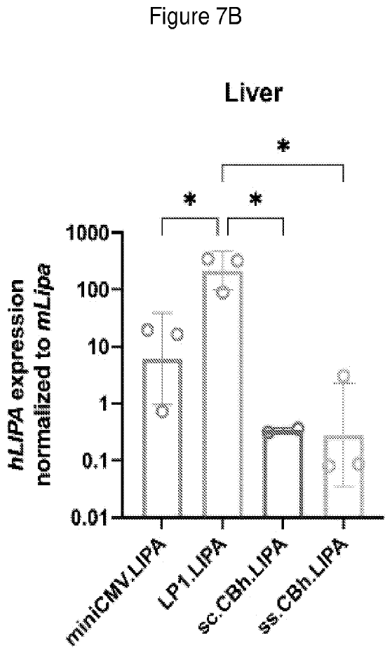

AAV vector genome (vg) biodistribution for all four AAV vectors, e.g., scAAV.miniCMV.LIPA, scAAV.LP1.LIPA, scAAV.Cbh.LIPA, and ssAAV.Cbh.LIPA, in tissues throughout the body was analyzed after intravenous (IV) dosing at 4×10¹³ vg/kg in Lipa^{-/-} mice (see FIG. 6). Vector genomes (vg) per nucleus were measured for four different AAV vectors after treatment of Lipa^{-/-} mice. Mice were treated intravenously (IV) at 2 months, with analysis at 6 months of age. Note that scAAV.miniCMV.LIPA transduction in the liver exceeds scAAV.LP1.LIPA by 10-fold, yet scAAV.LP1.LIPA will exceed LIPA gene expression in the liver relative to scAAV.miniCMV.LIPA by 36-fold (see FIG. 7). Also note the low transduction of both Cbh vectors (scAAV.Cbh.LIPA and ssAAV.Cbh.LIPA) in liver, suggesting immune clearance.

Both Cbh vectors (scAAV.Cbh.LIPA and ssAAV.Cbh.LIPA) showed almost no transduction in the liver at 4 months post-treatment (6 months of age), suggesting the transgene had been eliminated from the liver. Cbh vectors (scAAV.Cbh.LIPA and ssAAV.Cbh.LIPA) showed transduction equal to or exceeding scAAV.LP1.LIPA in other tissues (e.g., muscle). This suggest that the Cbh constructs (scAAV.Cbh.LIPA and ssAAV.Cbh.LIPA) were not given at an incorrect dose, but that they were actively eliminated from the liver of Lipa^{-/-} mice. The other note here is that the scAAV.LP1.LIPA vector showed slightly reduced transduction of all organs relative to scAAV.miniCMV.LIPA. Thus, the results suggest that the gene expression and therapeutic effects observed with scAAV.LP1.LIPA were not the result of more AAV vgs being present.

### Example 7

### LP1 Shows Improved Ability to Induce and Sustain LIPA Gene Expression in Liver

LIPA transgene expression, relative to endogenous mouse wild type Lipa gene expression, in various tissues of Lipa^{-/-} mice was analyzed. Mice were treated intravenously (IV) at 2 months, with analysis at 6 months of age. scAAV.LP1.LIPA induced gene expression in the liver is 216 times normal wild type mouse liver Lipa gene expression, while scAAV.miniCMV.LIPA induced gene expression is only 6 times normal. So scAAV.LP1.LIPA yields 36 times more gene expression in the liver, despite showing lower AAV transduction (see FIG. 6). scAAV.LP1.LIPA expression in other organs (e.g., muscle, lung, kidney, and heart) is quite low, consistent with the liver-specific nature of the LP1 promoter.

Thus, scAAV.LP1.LIPA showed a profoundly improved ability to induce and sustain LIPA gene expression in the liver. As expected, scAAV.LP1.LIPA also showed much lower LIPA gene expression in all non-liver tissues, perhaps except the lymph node. This data was consistent with the fact that LP1 is a “liver-specific” promoter. As known from previous studies of Factor VIII and Factor IX expression for Hemophilia gene therapy, confinement of gene expression to the liver can greatly suppress both B and T cell responses to the transgenic protein in animals where the endogenous gene has been completely knocked out (as is the present case).

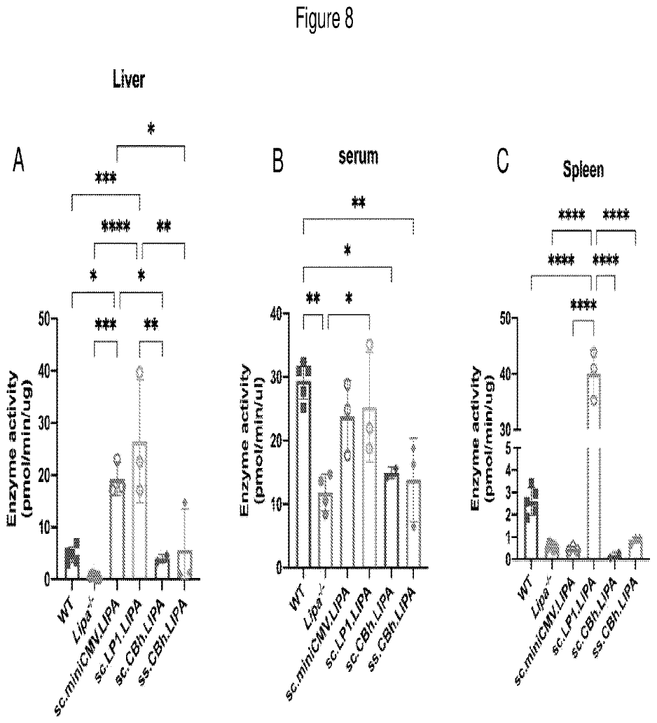

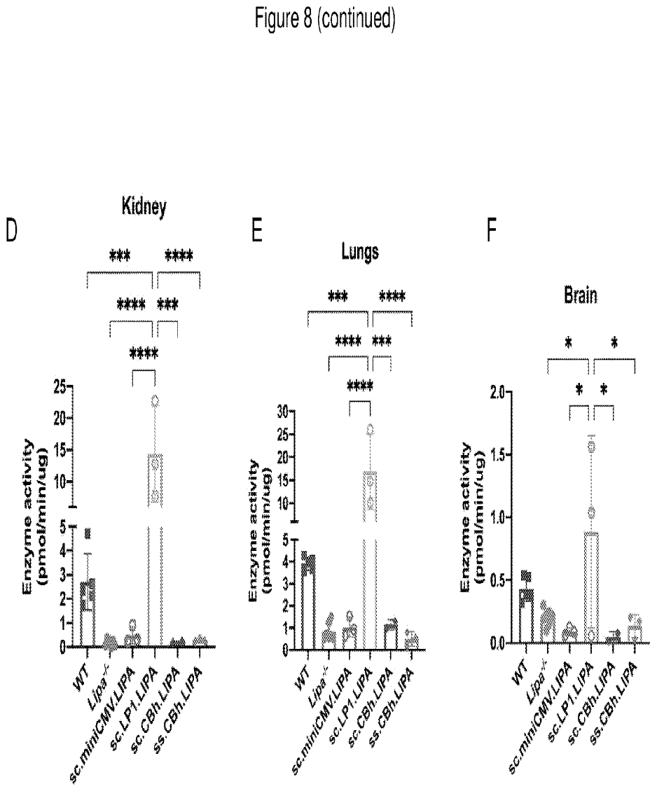

Because LP1 drove such high gene expression in the liver, it is suspected that this would induce increased LIPA enzyme secretion from the liver, which would be manifested as increased enzyme replacement (protein) therapy in non-liver organs. To test this, LIPA enzyme activities in kidney, lung, spleen, and brain in addition to liver and serum were analyzed. FIG. 8 shows LIPA enzyme activity in tissues after intravenous (IV) treatment of AAV vectors (e.g., scAAV.miniCMV.LIPA, scAAV.LP1.LIPA, scAAV.Cbh.LIPA, and ssAAV.Cbh.LIPA) in Lipa^{-/-} mice. Wild type (WT) and untreated Lipa^{-/-} mice were compared to Lipa^{-/-} treated with one of four AAVrh74 vectors (e.g., scAAV.miniCMV.LIPA, scAAV.LP1.LIPA, scAAV.Cbh.LIPA, and ssAAV.Cbh.LIPA) intravenously (IV) at 2 months of age at a dose of 4×10¹³ vg/kg. All mice were analyzed at 6 months of age, after 4 months of treatment. As expected, LIPA enzyme activities in liver and serum, while increased, was not increased as dramatically as gene expression. Without wishing to be bound by theory, it is possible that because the majority of overexpressed protein in liver will be secreted into the serum, and the majority of protein in the serum will be transported into other tissues. This is a common finding in studies of other lysosomal enzymes and as in studies of enzyme replacement therapy (ERT).

Enzyme activity in non-liver organs was dramatically increased by LP1. For example, LP1 induced LIPA enzyme activity (by scAAV.LP1.LIPA vector) in Lipa^{-/-} mouse spleen that was 16 times the normal wild type level (and 80 times the level found in untreated Lipa^{-/-} mice), while the inclusion of the miniCMV promoter (by scAAV.miniCMV.LIPA vector) did not induce any increase in activity relative to untreated Lipa^{-/-} mice. LP1-treated kidney (i.e., treated with scAAV.LP1.LIPA vector) had LAL activity that was 5.3 times wild type level, while scAAV.miniCMV.LIPA was 0.1 times wild type. scAAV.LP1.LIPA treated Lipa^{-/-} lung had 4.3 times wild type LAL activity, while scAAV.miniCMV.LIPA had 0.26 times wild type. Even in the brain, which one would expect LIPA protein not to cross from serum in high amounts, it was found that LP1-treated (scAAV.LP1.LIPA) Lipa^{-/-} brain had 2 times wild type activity, while scAAV.miniCMV.LIPA had 0.19 times wild type. Thus, the ability of LP1 promoter to dramatically increase LIPA gene expression in the liver was translated to more effective ERT therapy in non-expressing tissues. Moreover, the risk of autoimmune reactions in the liver with another constitutive promoter, e.g., Cbh (such as in vectors scAAV.Cbh.LIPA and ssAAV.Cbh.LIPA), suggests significant safety risks in using a constitutive promoter to drive LIPA gene therapy.

For the reasons stated in the above Examples, LP1 liver specific LIPA gene therapy for the treatment of LALD was chosen for further experiments and as a strong candidate for future therapies. Because LIPA enzyme activity is lowered in nonalcoholic fatty liver disease (NAFLD) and in nonalcoholic steatohepatitis (NASH), and is a biomarker where levels of reduction reflect increased disease severity, it is possible that this gene therapy may also be used to treat these disorders, as well as general disorders where triglycerides and cholesterol are elevated, including obesity and heart disease.

### Example 8

### Additional Investigation of Intravenous (IV) Treatments by Various Constructs at Different Time Points

Intravenous (IV) treatment of various AAVr74 vectors expressing LIPA were tested in Lipa^{-/-} mice. The treatments are summarized in the Table 3 below. Mice were dosed at 2 months of age, a time when disease is already severe, or at 2 days of age (denoted as “P2”) which is prior to significant disease onset. All mice were assayed at 6 months of age, 4 months after treatment, a point at which LAL-D disease is quite severe.

TABLE 3

| | | | Time of Injection |
|---|---|---|---|
| Treatment vector | Promoter | Dose | (age) |
| No treatment in wild | | | |
| type mice (wild type) | | | |
| No treatment in | | | |
| LIPA^{-/-} mice (LIPA^{-/-}) | | | |
| ssAAVrh74.Cbh.LIPA | CbH | 4.2e13 vg/kg | 2 months of age |
| scAAVrh74.Cbh.LIPA, | Cbh | 4.2e13 vg/kg | 2 months of age |
| scAAVrh74.LP1.LIPA | LP1 | 4.2e13 vg/kg | 2 months of age |
| scAAVrh74.LP1.LIPA | LP1 | 2.1e13 vg/kg | 2 months of age |
| scAAVrh74.LP1.LIPA | LP1 | 1.0e13 vg/kg | 2 months of age |
| scAAVrh74.LP1.LIPA | LP1 | 5.0e12 vg/kg | 2 months of age |
| scAAVrh74.LP1.LIPA | LP1 | 1.0e12 vg/kg | 2 months of age |
| scAAVrh74.LP1.LIPA | LP1 | 4.2e13 vg/kg | 2 days (P2) |
| scAAVrh74.LP1.LIPA | LP1 | 2.1e13 vg/kg | 2 days (P2) |

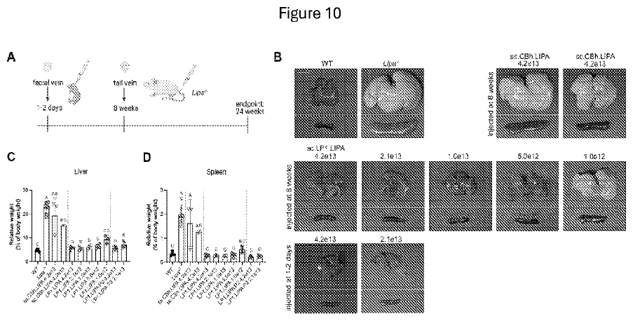

Hepatosplenomegaly was investigated at 6 months of age. As shown in FIG. 10, liver and spleen size increased dramatically in untreated Lipa^{-/-} mice relative to wild type, such that the liver is about 4.8 times normal size by 6 months of age. Treatment with AAV vectors having the LP1 promoter reduced the size of the liver and spleen, while both scAAV.Cbh.LIPA and ssAAV.Cbh.LIPA had less therapeutic impact. Treatment with the scAAV.LP1.LIPA was effective at all doses tested.

Lipid content in the livers from the treated Lipa^{-/-} mice was assayed by staining tissue section with oil O before and after injection. As shown in FIG. 11, treatment with rscAAVrh74.LP1.LIPA reduced lipid content in the liver of Lipa^{-/-} mice.

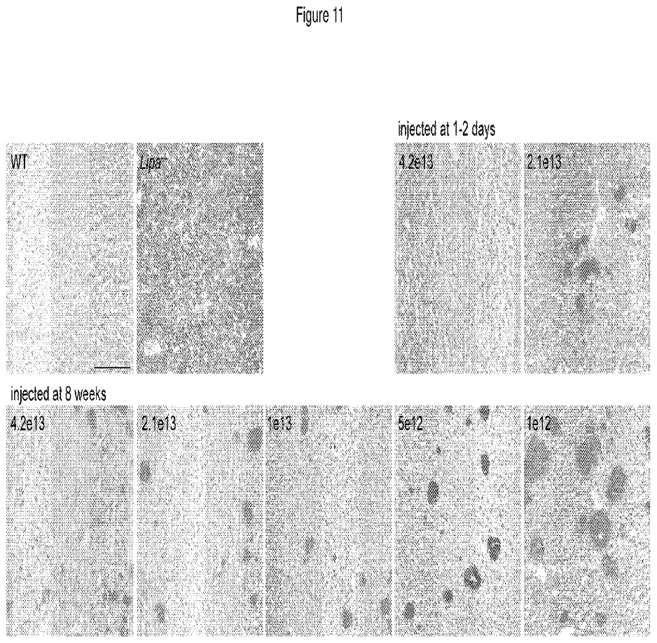

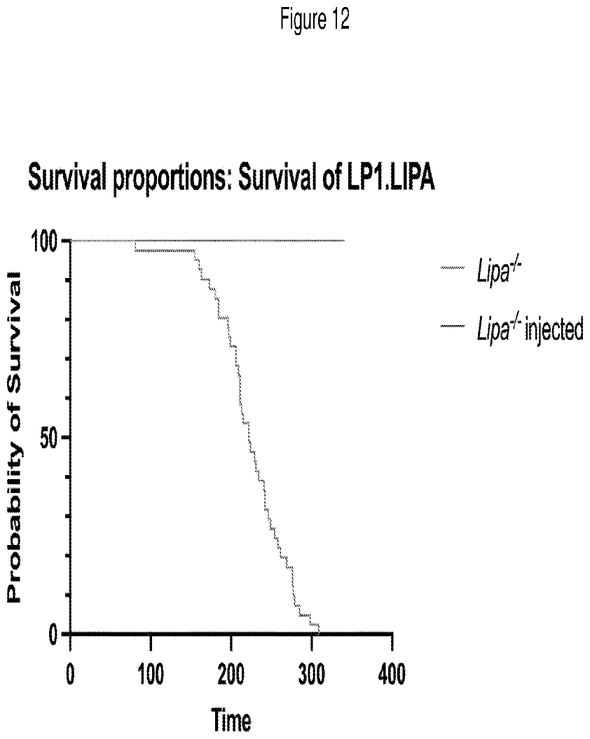

FIG. 12 demonstrates that IV administration of 1×10¹³ vg/kg of rscAAV.LP1.LIPA gene therapy increased survival of treated Lipa^{-/-} mice. Untreated Lipa^{-/-} mice do not survive beyond 305 days of age. Lipa^{-/-} mice treated at 2 months of age (60 days) all survive to beyond this time point.

AAV vector biodistribution and LIPA gene expression was measured in liver, spleen, intestine, and heart of the treated Lipa^{-/-} mice using the methods described in Example 6 above. Lipa^{-/-} mice were treated at 2 months or treated at postnatal day 2 (marked P2) and analyzed at 6 months of age. AAV vectors having a promoter that is optimized for constitutive gene expression in all tissues (Cbh) was compared to LP1, a liver-specific promoter. Cbh showed poor sustained gene expression in liver, while LP1 showed very strong gene expression in liver. Cbh also showsed lower AAV biodistribution in liver, suggesting clearance by immune factors. LP1 only showed high levels of gene overexpression in liver (see FIG. 13).

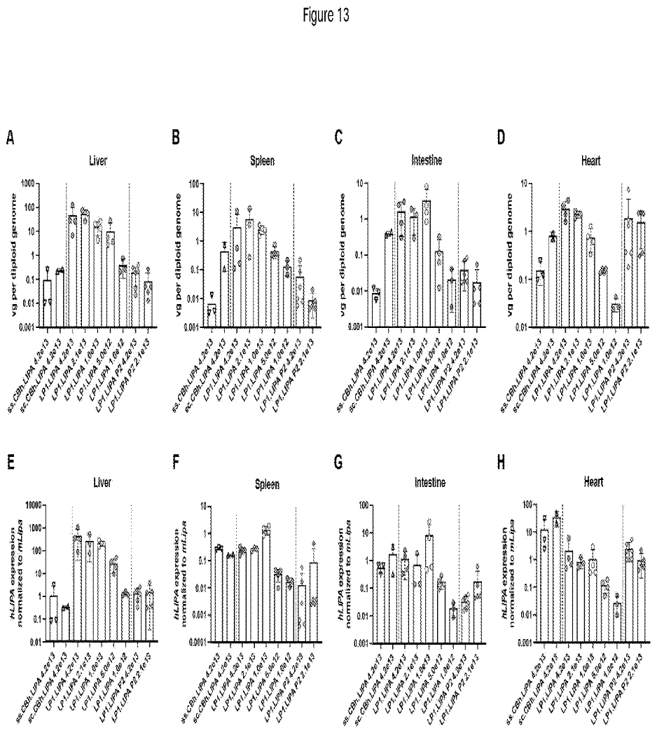

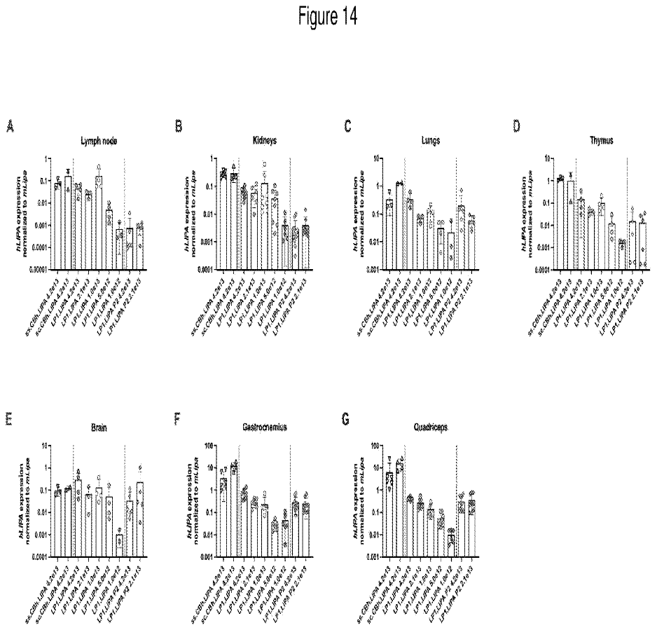

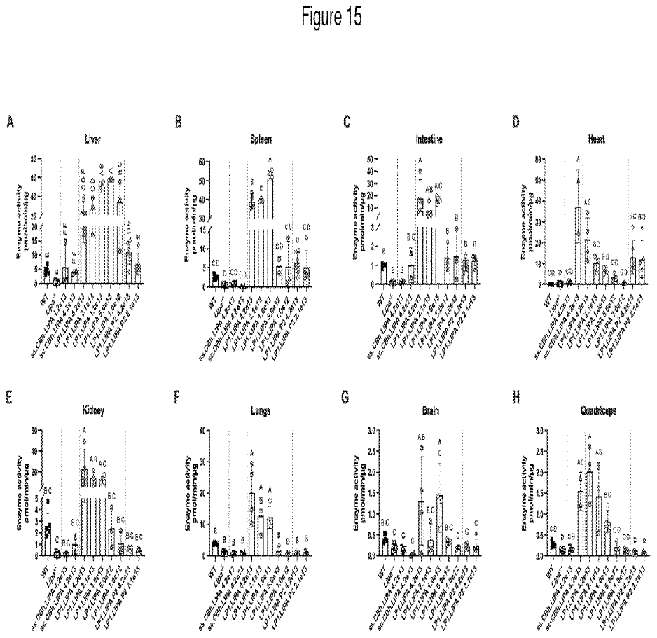

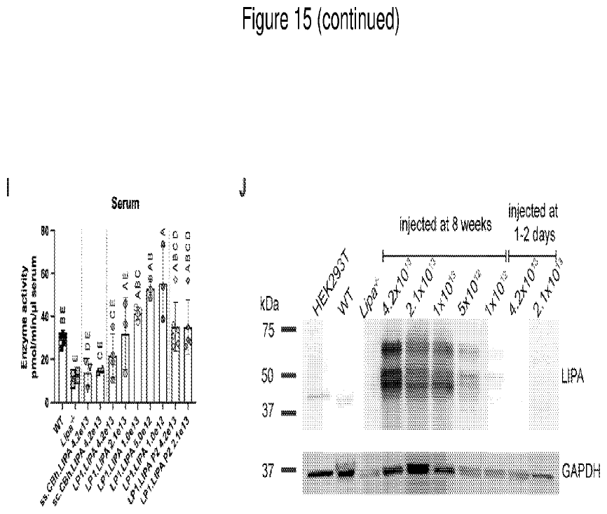

Treatment of the rscAAV.LP1.LIPA vector resulted in liver specific gene expression. As shown in FIG. 14, expression of LIPA driven by the LP1 promoter resulted in no detectable gene expression in the lymph node, lung, kidney, thymus, brain or skeletal muscles (gastrocnemius and quadriceps). However, IV administration of rscAAVrh74.LP1.LIPA results in cross correction of LP1 enzyme replacement therapy in non-expressing organs despite AAV-mediated LIPA expression being confined to the liver As shown in FIG. 15, LP1 enzyme activity was elevated in non-liver organs despite AAV-mediated LIPA gene expression being confined to the liver. This is evidence that cross-correction of LIPA protein from liver occurred in organs throughout the body, including spleen, intestine, heart, kidney, lungs, brain, skeletal muscle (quadriceps are shown), and serum, which can allow for therapeutic correction of disease throughout the body.

### REFERENCES

1. Pastores, G M, and Hughes, D A (2020). Lysosomal Acid Lipase Deficiency: Therapeutic Options. Drug Des Devel Ther 14: 591-601.
2. Gomaraschi, M, Bonacina, F, and Norata, G D (2019). Lysosomal Acid Lipase: From Cellular Lipid Handler to Immunometabolic Target. Trends Pharmacol Sci 40: 104-115.
3. Li, F, and Zhang, H (2019). Lysosomal Acid Lipase in Lipid Metabolism and Beyond. Arterioscler Thromb Vasc Biol 39: 850-856.
4. Aguisanda, F, Thorne, N, and Zheng, W (2017). Targeting Wolman Disease and Cholesteryl Ester Storage Disease: Disease Pathogenesis and Therapeutic Development. Curr Chem Genom Transl Med 11: 1-18.
5. Abramov, A, Schorr, S, and Wolman, M (1956). Generalized xanthomatosis with calcified adrenals. AMA J Dis Child 91: 282-286.
6. Pericleous, M, Kelly, C, Wang, T, Livingstone, C, and Ala, A (2017). Wolman's disease and cholesteryl ester storage disorder: the phenotypic spectrum of lysosomal acid lipase deficiency. Lancet Gastroenterol Hepatol 2: 670-679.
7. Shan, Z, and Ju, C (2020). Hepatic Macrophages in Liver Injury. Front Immunol 11: 322.
8. Baratta, F, Pastori, D, Ferro, D, Carluccio, G, Tozzi, G, Angelico, F, et al. (2019). Reduced lysosomal acid lipase activity: A new marker of liver disease severity across the clinical continuum of non-alcoholic fatty liver disease? World J Gastroenterol 25: 4172-4180.
9. Du, H, and Grabowski, G A (2004). Lysosomal acid lipase and atherosclerosis. Curr Opin Lipidol 15: 539-544.
10. Wild, P S, Zeller, T, Schillert, A, Szymczak, S, Sinning, C R, Deiseroth, A, et al. (2011). A genome-wide association study identifies LIPA as a susceptibility gene for coronary artery disease. Circ Cardiovasc Genet 4: 403-412.
11. Burton, B K, Balwani, M, Feillet, F, Baric, I, Burrow, T A, Camarena Grande, C, et al. (2015). A Phase 3 Trial of Sebelipase Alfa in Lysosomal Acid Lipase Deficiency. N Engl J Med 373: 1010-1020.
12. Spencer, H T, Riley, B E, and Doering, C B (2016). State of the art: gene therapy of haemophilia. Haemophilia 22 Suppl 5: 66-71.
13. High, K A (2012). The gene therapy journey for hemophilia: are we there yet? Hematology Am Soc Hematol Educ Program 2012: 375-381.
14. Rodino-Klapac, L R, Chicoine, L G, Kaspar, B K, and Mendell, J R (2007). Gene therapy for duchenne muscular dystrophy: expectations and challenges. Arch Neurol 64: 1236-1241.
15. Mendell, J R, Al-Zaidy, S, Shell, R, Arnold, W D, Rodino-Klapac, L R, Prior, T W, et al. (2017). Single-Dose Gene-Replacement Therapy for Spinal Muscular Atrophy. N Engl J Med 377: 1713-1722.
16. Testa, F, Maguire, A M, Rossi, S, Pierce, E A, Melillo, P, Marshall, K, et al. (2013). Three-year follow-up after unilateral subretinal delivery of adeno-associated virus in patients with Leber congenital Amaurosis type 2. Ophthalmology 120: 1283-1291.
17. Nagree, M S, Scalia, S, McKillop, W M, and Medin, J A (2019). An update on gene therapy for lysosomal storage disorders. Expert Opin Biol Ther 19: 655-670.
18. Penati, R, Fumagalli, F, Calbi, V, Bernardo, M E, and Aiuti, A (2017). Gene therapy for lysosomal storage disorders: recent advances for metachromatic leukodystrophy and mucopolysaccaridosis I. J Inherit Metab Dis 40: 543-554.
19. Biffi, A (2016). Gene therapy for lysosomal storage disorders: a good start. Hum Mol Genet 25: R65-75.
20. Rastall, D P, and Amalfitano, A (2015). Recent advances in gene therapy for lysosomal storage disorders. Appl Clin Genet 8: 157-169.
21. Chicoine, L G, Rodino-Klapac, L R, Shao, G, Xu, R, Bremer, W G, Camboni, M, et al. (2014). Vascular delivery of rAAVrh74.MCK.GALGT2 to the gastrocnemius muscle of the rhesus macaque stimulates the expression of dystrophin and laminin alpha2 surrogates. Mol Ther 22: 713-724.
22. Martin, P T, Xu, R, Rodino-Klapac, L R, Oglesbay, E, Camboni, M, Montgomery, C L, et al. (2009). Overexpression of Galgt2 in skeletal muscle prevents injury resulting from eccentric contractions in both mdx and wild-type mice. Am J Physiol Cell Physiol 296: C476-488.
23. Grieger, J C, and Samulski, R J (2005). Adeno-associated virus as a gene therapy vector: vector development, production and clinical applications. Adv Biochem Eng Biotechnol 99: 119-145.
24. Rosas, L E, Grieves, J L, Zaraspe, K, La Perle, K M, Fu, H, and McCarty, D M (2012). Patterns of scAAV vector insertion associated with oncogenic events in a mouse model for genotoxicity. Mol Ther 20: 2098-2110.
25. Xiao, X, Li, J, and Samulski, R J (1998). Production of high-titer recombinant adeno-associated virus vectors in the absence of helper adenovirus. J Virol 72: 2224-2232.
26. Bish, L T, Morine, K, Sleeper, M M, Sanmiguel, J, Wu, D, Gao, G, et al. (2008). Adeno-associated virus (AAV) serotype 9 provides global cardiac gene transfer superior to AAV1, AAV6, AAV7, and AAV8 in the mouse and rat. Hum Gene Ther 19:1359-1368.
27. Cunningham, S C, and Alexander, I E (2019). AAV-Mediated Gene Delivery to the Mouse Liver. Methods Mol Biol 1937: 213-219.
28. Palaschak, B, Herzog, R W, and Markusic, D M (2019). AAV-Mediated Gene Delivery to the Liver: Overview of Current Technologies and Methods. Methods Mol Biol 1950: 333-360.
29. McCarty, D M (2008). Self-complementary AAV vectors; advances and applications. Mol Ther 16: 1648-1656.
30. Du, H, Duanmu, M, Witte, D, and Grabowski, G A (1998). Targeted disruption of the mouse lysosomal acid lipase gene: long-term survival with massive cholesteryl ester and triglyceride storage. Hum Mol Genet 7: 1347-1354.
31. Du, H, Heur, M, Duanmu, M, Grabowski, G A, Hui, D Y, Witte, D P, et al. (2001). Lysosomal acid lipase-deficient mice: depletion of white and brown fat, severe hepatosplenomegaly, and shortened life span. J Lipid Res 42: 489-500.
32. Sun, Y, Xu, Y H, Du, H, Quinn, B, Liou, B, Stanton, L, et al. (2014). Reversal of advanced disease in lysosomal acid lipase deficient mice: a model for lysosomal acid lipase deficiency disease. Mol Genet Metab 112: 229-241.
33. Lam, P., Ashbrook, A., Zygmunt, D. A., Yang, C., Du, H., and Martin, P. T. (2022) Therapeutic efficacy of rscAAVrh74.miniCMV.LIPA gene therapy in a mouse model of lysosomal acid lipase deficiency. Mol. Ther. Methods Clin Devel. 26:413-426.

### Sequences

### Nucleotide Sequence of LIPA Gene Variant 1 (SEQ ID NO: 1)

ATGAAAATGCGGTTCTTGGGGTTGGTGGTCTGTTTGGTTCTCTGGACCCTGCATTCTGAGGGGTCTGG
AGGGAAACTGACAGCTGTGGATCCTGAAACAAACATGAATGTGAGTGAAATTATCTCTTACTGGGGAT
TCCCTAGTGAGGAATACCTAGTTGAGACAGAAGATGGATATATTCTGTGCCTTAACCGAATTCCTCAT
GGGAGGAAGAACCATTCTGACAAAGGTCCCAAACCAGTTGTCTTCCTGCAACATGGCTTGCTGGCAGA
TTCTAGTAACTGGGTCACAAACCTTGCCAACAGCAGCCTGGGCTTCATTCTTGCTGATGCTGGTTTTG
ACGTGTGGATGGGCAACAGCAGAGGAAATACCTGGTCTCGGAAACATAAGACACTCTCAGTTTCTCAG
GATGAATTCTGGGCTTTCAGTTATGATGAGATGGCAAAATATGACCTACCAGCTTCCATTAACTTCAT
TCTGAATAAAACTGGCCAAGAACAAGTGTATTATGTGGGTCATTCTCAAGGCACCACTATAGGTTTTA
TAGCATTTTCACAGATCCCTGAGCTGGCTAAAAGGATTAAAATGTTTTTTGCCCTGGGTCCTGTGGCT
TCCGTCGCCTTCTGTACTAGCCCTATGGCCAAATTAGGACGATTACCAGATCATCTCATTAAGGACTT
ATTTGGAGACAAAGAATTTCTTCCCCAGAGTGCGTTTTTGAAGTGGCTGGGTACCCACGTTTGCACTC
ATGTCATACTGAAGGAGCTCTGTGGAAATCTCTGTTTTCTTCTGTGTGGATTTAATGAGAGAAATTTA
AATATGTCTAGAGTGGATGTATATACAACACATTCTCCTGCTGGAACTTCTGTGCAAAACATGTTACA
CTGGAGCCAGGCTGTTAAATTCCAAAAGTTTCAAGCCTTTGACTGGGGAAGCAGTGCCAAGAATTATT
TTCATTACAACCAGAGTTATCCTCCCACATACAATGTGAAGGACATGCTTGTGCCGACTGCAGTCTGG
AGCGGGGGTCACGACTGGCTTGCAGATGTCTACGACGTCAATATCTTACTGACTCAGATCACCAACTT
GGTGTTCCATGAGAGCATTCCGGAATGGGAGCATCTTGACTTCATTTGGGGCCTGGATGCCCCTTGGA
GGCTTTATAATAAAATTATTAATCTAATGAGGAAATATCAGTGA

### Amino Acid Sequence of LIPA Gene Variant 1 (SEQ ID NO: 2)

MKMRFLGLVVCLVLWTLHSEGSGGKLTAVDPETNMNVSEIISYWGFPSEEYLVETEDGYILCLNRIPH
GRKNHSDKGPKPVVFLOHGLLADSSNWVTNLANSSLGFILADAGFDVWMGNSRGNTWSRKHKTLSVSQ
DEFWAFSYDEMAKYDLPASINFILNKTGQEQVYYVGHSQGTTIGFIAFSQIPELAKRIKMFFALGPVA
SVAFCTSPMAKLGRLPDHLIKDLFGDKEFLPQSAFLKWLGTHVCTHVILKELCGNLCELLCGFNERNL
NMSRVDVYTTHSPAGTSVQNMLHWSQAVKFQKFQAFDWGSSAKNYFHYNQSYPPTYNVKDMLVPTAVW
SGGHDWLADVYDVNILLTQITNLVFHESIPEWEHLDFIWGLDAPWRLYNKIINLMRKYQ

### Sequence of LP1 Promoter (SEQ ID NO: 3)

CCCTAAAATGGGCAAACATTGCAAGCAGCAAACAGCAAACACACAGCCCTCCCTGCCTGCTGACCTTG
GAGCTGGGGCAGAGGTCAGAGACCTCTCTGGGCCCATGCCACCTCCAACATCCACTCGACCCCTTGGA
ATTTCGGTGGAGAGGAGCAGAGGTTGTCCTGGCGTGGTTTAGGTAGTGTGAGAGGGGAATGACTCCTT
TCGGTAAGTGCAGTGGAAGCTGTACACTGCCCAGGCAAAGCGTCCGGGCAGCGTAGGCGGGCGACTCA
GATCCCAGCCAGTGGACTTAGCCCCTGTTTGCTCCTCCGATAACTGGGGTGACCTTGGTTAATATTCA
CCAGCAGCCTCCCCCGTTGCCCCTCTGGATCCACTGCTTAAATACGGACGAGGACAGGGCCCTGTCTC
CTCAGCTTCAGGCACCACCACTGACCTGGGACAGTGAATC

### Sc.Ptrs.LP1.LIPA AAV Vector Plasmid Sequence (SEQ ID NO: 4)

ACCCAACTGATCTTCAGCATCTTTTACTTTCACCAGCGTTTCTGGGTGAGCAAAAACAGGAAGGCAAA
ATGCCGCAAAAAAGGGAATAAGGGCGACACGGAAATGTTGAATACTCATACTCTTCCTTTTTCAATAT
TATTGAAGCATTTATCAGGGTTATTGTCTCATGAGCGGATACATATTTGAATGTATTTAGAAAAATAA
ACAAATAGGGGTTCCGCGCACATTTCCCCGAAAAGTGCCACCTGACGTCTAAGAAACCATTATTATCA
TGACATTAACCTATAAAAATAGGCGTATCACGAGGCCCTTTCGTCTCGCGCGTTTCGGTGATGACGGT
GAAAACCTCTGACACATGCAGCTCCCGGAGACGGTCACAGCTTGTCTGTAAGCGGATGCCGGGAGCAG
ACAAGCCCGTCAGGGCGCGTCAGCGTGTGTTGGCGGGTGTCGGGGCTGGCTTAACTATGCGGCATCAG
AGCAGATTGTACTGAGAGTGCACCATATGCGGTGTGAAATACCGCACAGATGCGTAAGGAGAAAATAC
CGCATCAGGCGATTCCAACATCCAATAAATCATACAGGCAAGGCAAAGAATTAGCAAAATTAAGCAAT
AAAGCCTCAGAGCATAAAGCTAAATCGGTTGTACCAAAAACATTATGACCCTGTAATACTTTTGCGGG
AGAAGCCTTTATTTCAACGCAAGGATAAAAATTTTTAGAACCCTCATATATTTTAAATGCAATGCCTG
AGTAATGTGTAGGTAAAGATTCAAACGGGTGAGAAAGGCCGGAGACAGTCAAATCACCATCAATATGA
TATTCAACCGTTCTAGCTGATAAATTCATGCCGGAGAGGGTAGCTATTTTTGAGAGGTCTCTACAAAG
GCTATCAGGTCATTGCCTGAGAGTCTGGAGCAAACAAGAGAATCGATGAACGGTAATCGTAAAACTAG
CATGTCAATCATATGTACCCCGGTTGATAATCAGAAAAGCCCCAAAAACAGGAAGATTGTATAAGCAA
ATATTTAAATTGTAAGCGTTAATATTTTGTTAAAATTCGCGTTAAATTTTTGTTAAATCAGCTCATTT
TTTAACCAATAGGCCGAAATCGGCAAAATCCCTTATAAATCAAAAGAATAGACCGAGATAGGGTTGAG
TGTTGTTCCAGTTTGGAACAAGAGTCCACTATTAAAGAACGTGGACTCCAACGTCAAAGGGCGAAAAA
CCGTCTATCAGGGCGATGGCCCACTACGTGAACCATCACCCTAATCAAGTTTTTTGGGGTCGAGGTGC
CGTAAAGCACTAAATCGGAACCCTAAAGGGAGCCCCCGATTTAGAGCTTGACGGGGAAAGCCGGCGAA
CGTGGCGAGAAAGGAAGGGAAGAAAGCGAAAGGAGCGGGCGCTAGGGCGCTGGCAAGTGTAGCGGTCA
CGCTGCGCGTAACCACCACACCCGCCGCGCTTAATGCGCCGCTACAGGGCGCGTACTATGGTTGCTTT
GACGAGCACGTATAACGTGCTTTCCTCGTTAGAATCAGAGCGGGAGCTAAACAGGAGGCCGATTAAAG
GGATTTTAGACAGGAACGGTACGCCAGAATCCTGAGAAGTGTTTTTATAATCAGTGAGGCCACCGAGT
AAAAGAGTCTGTCCATCACGCAAATTAACCGTTGTCGCAATACTTCTTTGATTAGTAATAACATCACT
TGCCTGAGTAGAAGAACTCAAACTATCGGCCTTGCTGGTAATATCCAGAACAATATTACCGCCAGCCA
TTGCAACGGAATCGCCATTCGCCATTCAGGCTGCGCAACTGTTGGGAAGGGCGATCGGTGCGGGCCTC
TTCGCTATTACGCCAGCTGCGCGCTCGCTCGCTCACTGAGGCCGCCCGGGCAAAGCCCGGGCGTCGGG
CGACCTTTGGTCGCCCGGCCTCAGTGAGCGAGCGAGCGCGCAGAGAGGGAGTGGCCAACTCCATCACT
AGGGGTTCCTAGGAAGCTTCCCTAAAATGGGCAAACATTGCAAGCAGCAAACAGCAAACACACAGCCC
TCCCTGCCTGCTGACCTTGGAGCTGGGGCAGAGGTCAGAGACCTCTCTGGGCCCATGCCACCTCCAAC
ATCCACTCGACCCCTTGGAATTTCGGTGGAGAGGAGCAGAGGTTGTCCTGGCGTGGTTTAGGTAGTGT
GAGAGGGGAATGACTCCTTTCGGTAAGTGCAGTGGAAGCTGTACACTGCCCAGGCAAAGCGTCCGGGC
AGCGTAGGCGGGCGACTCAGATCCCAGCCAGTGGACTTAGCCCCTGTTTGCTCCTCCGATAACTGGGG
TGACCTTGGTTAATATTCACCAGCAGCCTCCCCCGTTGCCCCTCTGGATCCACTGCTTAAATACGGAC
GAGGACAGGGCCCTGTCTCCTCAGCTTCAGGCACCACCACTGACCTGGGACAGTGAATCCGGACTCTA
AGGTAAATATAAAATTTTTAAGTGTATAATGTGTTAAACTACTGATTCTAATTGTTTCTCTCTTTTAG
ATTCCAACCTTTGGAACTGAGGTACCGGCCGCTAGCCGCCACCATGAAAATGCGGTTCTTGGGGTTGG
TGGTCTGTTTGGTTCTCTGGACCCTGCATTCTGAGGGGTCTGGAGGGAAACTGACAGCTGTGGATCCT
GAAACAAACATGAATGTGAGTGAAATTATCTCTTACTGGGGATTCCCTAGTGAGGAATACCTAGTTGA
GACAGAAGATGGATATATTCTGTGCCTTAACCGAATTCCTCATGGGAGGAAGAACCATTCTGACAAAG
GTCCCAAACCAGTTGTCTTCCTGCAACATGGCTTGCTGGCAGATTCTAGTAACTGGGTCACAAACCTT
GCCAACAGCAGCCTGGGCTTCATTCTTGCTGATGCTGGTTTTGACGTGTGGATGGGCAACAGCAGAGG
AAATACCTGGTCTCGGAAACATAAGACACTCTCAGTTTCTCAGGATGAATTCTGGGCTTTCAGTTATG
ATGAGATGGCAAAATATGACCTACCAGCTTCCATTAACTTCATTCTGAATAAAACTGGCCAAGAACAA
GTGTATTATGTGGGTCATTCTCAAGGCACCACTATAGGTTTTATAGCATTTTCACAGATCCCTGAGCT
GGCTAAAAGGATTAAAATGTTTTTTGCCCTGGGTCCTGTGGCTTCCGTCGCCTTCTGTACTAGCCCTA
TGGCCAAATTAGGACGATTACCAGATCATCTCATTAAGGACTTATTTGGAGACAAAGAATTTCTTCCC
CAGAGTGCGTTTTTGAAGTGGCTGGGTACCCACGTTTGCACTCATGTCATACTGAAGGAGCTCTGTGG
AAATCTCTGTTTTCTTCTGTGTGGATTTAATGAGAGAAATTTAAATATGTCTAGAGTGGATGTATATA
CAACACATTCTCCTGCTGGAACTTCTGTGCAAAACATGTTACACTGGAGCCAGGCTGTTAAATTCCAA
AAGTTTCAAGCCTTTGACTGGGGAAGCAGTGCCAAGAATTATTTTCATTACAACCAGAGTTATCCTCC
CACATACAATGTGAAGGACATGCTTGTGCCGACTGCAGTCTGGAGCGGGGGTCACGACTGGCTTGCAG
ATGTCTACGACGTCAATATCTTACTGACTCAGATCACCAACTTGGTGTTCCATGAGAGCATTCCGGAA
TGGGAGCATCTTGACTTCATTTGGGGCCTGGATGCCCCTTGGAGGCTTTATAATAAAATTATTAATCT
AATGAGGAAATATCAGTGAGCATGCACTAGTGCGGCCGCGGATCTCAGACATGATAAGATACATTGAT
GAGTTTGGACAAACCACAACTAGAATGCAGTGAAAAAAATGCTTTATTTGTGAAATTTGTGATGCTAT
TGCTTTATTTGTAACCATTATAAGCTGCAATAAACAAGTTAACAACAACAATTGCATTCATTTTATGT
TTCAGGTTCAGGGGGAGGTGTGGGAGGTTTTTTAGGTTTAAACCCCACTCCCTCTCTGCGCGCTCGCT
CGCTCACTGAGGCCGGGCGACCAAAGGTCGCCCGACGCCCGGGCTTTGCCCGGGCGGCCTCAGTGAGC
GAGCGAGCGCGCAGCTGCATTAATGAATCGGCCAACGCGCGGGGAGAGGCGGTTTGCGTATTGGGCGC
TCTTCCGCTTCCTCGCTCACTGACTCGCTGCGCTCGGTCGTTCGGCTGCGGCGAGCGGTATCAGCTCA
CTCAAAGGCGGTAATACGGTTATCCACAGAATCAGGGGATAACGCAGGAAAGAACATGTGAGCAAAAG
GCCAGCAAAAGGCCAGGAACCGTAAAAAGGCCGCGTTGCTGGCGTTTTTCCATAGGCTCCGCCCCCCT
GACGAGCATCACAAAAATCGACGCTCAAGTCAGAGGTGGCGAAACCCGACAGGACTATAAAGATACCA
GGCGTTTCCCCCTGGAAGCTCCCTCGTGCGCTCTCCTGTTCCGACCCTGCCGCTTACCGGATACCTGT
CCGCCTTTCTCCCTTCGGGAAGCGTGGCGCTTTCTCATAGCTCACGCTGTAGGTATCTCAGTTCGGTG
TAGGTCGTTCGCTCCAAGCTGGGCTGTGTGCACGAACCCCCCGTTCAGCCCGACCGCTGCGCCTTATC
CGGTAACTATCGTCTTGAGTCCAACCCGGTAAGACACGACTTATCGCCACTGGCAGCAGCCACTGGTA
ACAGGATTAGCAGAGCGAGGTATGTAGGCGGTGCTACAGAGTTCTTGAAGTGGTGGCCTAACTACGGC
TACACTAGAAGGACAGTATTTGGTATCTGCGCTCTGCTGAAGCCAGTTACCTTCGGAAAAAGAGTTGG
TAGCTCTTGATCCGGCAAACAAACCACCGCTGGTAGCGGTGGTTTTTTTGTTTGCAAGCAGCAGATTA
CGCGCAGAAAAAAAGGATCTCAAGAAGATCCTTTGATCTTTTCTACGGGGTCTGACGCTCAGTGGAAC
GAAAACTCACGTTAAGGGATTTTGGTCATGAACAATAAAACTGTCTGCTTACATAAACAGTAATACAA
GGGGTGTTATGAGCCATATTCAACGGGAAACGTCTTGCTCTAGGCCGCGATTAAATTCCAACATGGAT
GCTGATTTATATGGGTATAAATGGGCTCGCGATAATGTCGGGCAATCAGGTGCGACAATCTATCGATT
GTATGGGAAGCCCGATGCGCCAGAGTTGTTTCTGAAACATGGCAAAGGTAGCGTTGCCAATGATGTTA
CAGATGAGATGGTCAGACTAAACTGGCTGACGGAATTTATGCCTCTTCCGACCATCAAGCATTTTATC
CGTACTCCTGATGATGCATGGTTACTCACCACTGCGATCCCCGGGAAAACAGCATTCCAGGTATTAGA
AGAATATCCTGATTCAGGTGAAAATATTGTTGATGCGCTGGCAGTGTTCCTGCGCCGGTTGCATTCGA
TTCCTGTTTGTAATTGTCCTTTTAACAGCGATCGCGTATTTCGTCTCGCTCAGGCGCAATCACGAATG
AATAACGGTTTGGTTGATGCGAGTGATTTTGATGACGAGCGTAATGGCTGGCCTGTTGAACAAGTCTG
GAAAGAAATGCATAAACTTTTGCCATTCTCACCGGATTCAGTCGTCACTCATGGTGATTTCTCACTTG
ATAACCTTATTTTTGACGAGGGGAAATTAATAGGTTGTATTGATGTTGGACGAGTCGGAATCGCAGAC
CGATACCAGGATCTTGCCATCCTATGGAACTGCCTCGGTGAGTTTTCTCCTTCATTACAGAAACGGCT
TTTTCAAAAATATGGTATTGATAATCCTGATATGAATAAATTGCAGTTTCATTTGATGCTCGATGAGT
TTTTCTAAGAATTCGTTCTTCGGGGCGAAAACTCTCAAGGATCTTACCGCTGTTGAGATCCAGTTCGA
TGTAACCCACTCGTGC

What is claimed is:

1. A polynucleotide comprising
(a) a liver-specific regulatory control element; and
(b) a lipase A (LIPA) cDNA sequence.

2. The polynucleotide of claim 1, wherein the liver-specific regulatory control element is a liver-specific promoter or a fragment thereof.

3. The polynucleotide of claim 2, wherein the liver-specific promoter is an LP1 promoter.

4. The polynucleotide of any one of claims 1-3 further comprising the nucleotide sequence of SEQ ID NO: 3.

5. The polynucleotide of any one of claims 1-4, wherein the LIPA cDNA comprises (a) a nucleotide sequence having at least 95% sequence identity to SEQ ID NO: 1 or (b) the nucleotide sequence set forth in SEQ ID NO: 1.

6. The polynucleotide of any one of claims 1-5 comprising (a) a nucleotide sequence that is at least 95% identical to nucleotides 1853-4094 of SEQ ID NO: 4 or (b) the nucleotide sequence of nucleotides 1853-4094 of SEQ ID NO: 4.

7. A recombinant adeno-associated virus (rAAV) having a genome comprising a polynucleotide sequence of any one of claims 1-6.

8. The rAAV of claim 7, wherein the rAAV is of the serotype AAV1, AAV2, AAV3, AAV4, AAV5, AAV6, AAV7, AAV8, AAV9, AAVRH10, AAVrh74, AAV11, AAV12, AAV13, Anc80, AAV7m8, or any derivative thereof.

9. The rAAV of claim 8, wherein the rAAV is of the serotype AAVrh74.

10. An rAAV particle comprising the rAAV of any one of claims 7-9.

11. A composition comprising the rAAV of any one of claims 7-9 or the viral particle of claim 10.

12. The composition of claim 11, wherein the composition is formulated for intravenous delivery.

13. A method of treating a Lysosomal Acid Lipase Deficiency (LAL-D) in a subject in need thereof comprising administering a polynucleotide of any one of claims 1-6, an rAAV of any one of claims 7-9, an rAAV particle of claim 10, or a composition of claim 11 or 12.

14. The method of claim 13 wherein the LAL-D is Wolman disease or cholesteryl ester storage disease (CESD).

15. The method of claim 13 or 14 wherein the subject has a mutation in the LAL-D gene.

16. A method of treating a disorder related to lipid storage or accumulation in a subject in need thereof comprising administering a polynucleotide of any one of claims 1-6, an rAAV of any one of claims 7-9, an rAAV particle of claim 10, or a composition of claim 11 or 12.

17. The method of claim 16, wherein the disorder is coronary artery disease, atherosclerosis, type II diabetes, obesity, nonalcoholic fatty liver disease (NAFLD), or nonalcoholic steatohepatitis (NASH).

18. A method of treating dyslipidemia or hypercholesterolemia in a subject in need thereof comprising administering a polynucleotide of any one of claims 1-6, an rAAV of any one of claims 7-9, an rAAV particle of claim 10, or a composition of claim 11 or 12.

19. A method of decreasing triglycerides, cholesterol, and/or fatty acids in a subject in need thereof comprising administering a polynucleotide of any one of claims 1-6, an rAAV of any one of claims 7-9, an rAAV particle of claim 10, or a composition of claim 11 or 12.

20. The method of any one of claims 13-19, further comprising a step of administering an immunosuppressing agent.

21. The method of any one of claims 13-19, wherein the polynucleotide, rAAV, rAAV particle, or composition are administered by intravenous or intraperitoneal delivery.

22. Use of a polynucleotide of any one of claims 1-6, an rAAV of any one of claims 7-9, an rAAV particle of claim 10, or a composition of claim 11 or 12 for the preparation of a medicament for the treatment of a Lysosomal Acid Lipase Deficiency (LAL-D) in a subject in need thereof.

23. The use of claim 22 wherein the LAL-D is Wolman disease or cholesteryl ester storage disease (CESD).

24. The use of claim 22 or 23 wherein the subject has a mutation in the LAL-D gene.

25. Use of a polynucleotide of any one of claims 1-6, an rAAV of any one of claims 7-9, an rAAV particle of claim 10, or a composition of claim 11 or 12 for the preparation of a medicament for the treatment of a disorder related to lipid storage or accumulation in a subject in need thereof.

26. The use of claim 25 wherein the disorder is coronary artery disease, atherosclerosis, type II diabetes, obesity, nonalcoholic fatty liver disease (NAFLD), or nonalcoholic steatohepatitis (NASH).

27. Use of a polynucleotide of any one of claims 1-6, an rAAV of any one of claims 7-9, an rAAV particle of claim 10, or a composition of claim 11 or 12 for the preparation of a medicament for treating dyslipidemia or hypercholesterolemia in a subject in need thereof.

28. Use of a polynucleotide of any one of claims 1-6, an rAAV of any one of claims 7-9, an rAAV particle of claim 10, or a composition of claim 11 or 12 for the preparation of a medicament for decreasing triglycerides, cholesterol, and/or fatty acids in a subject in need thereof.

29. The use of any one of claims 22-28, wherein the medicament is administered with an immunosuppressing agent.

30. The use of any one of claims 22-29, wherein the medicament is formulated for intravenous or intraperitoneal delivery.

31. A composition for treating a Lysosomal Acid Lipase Deficiency (LAL-D) in a subject in need thereof comprising, wherein the composition comprises a polynucleotide of any one of claims 1-6, an rAAV of any one of claims 7-9, an rAAV particle of claim 10, or a composition of claim 11 or 12.

32. The composition of claim 31 wherein the LAL-D is Wolman disease or cholesteryl ester storage disease (CESD).

33. The composition of claim 31 or 32 wherein the subject has a mutation in the LAL-D gene.

34. A composition for treating a disorder related to lipid storage or accumulation in a subject in need thereof, wherein the composition comprises a polynucleotide of any one of claims 1-6, an rAAV of any one of claims 7-9, an rAAV particle of claim 10, or a composition of claim 11 or 12.

35. The composition of claim 34 wherein the disorder is coronary artery disease, atherosclerosis, type II diabetes, obesity, nonalcoholic fatty liver disease (NAFLD), or nonalcoholic steatohepatitis (NASH).

36. A composition for treating dyslipidemia or hypercholesterolemia in a subject in need thereof, wherein the composition comprises a polynucleotide of any one of claims 1-6, an rAAV of any one of claims 7-9, an rAAV particle of claim 10, or a composition of claim 11 or 12.

37. A composition for decreasing triglycerides, cholesterol, and/or fatty acids in a subject in need thereof, wherein the composition comprises a polynucleotide of any one of claims 1-6, an rAAV of any one of claims 7-9, an rAAV particle of claim 10, or a composition of claim 11 or 12.

38. The composition of any one of claims 31-37, wherein the composition is administered with an immunosuppressing agent.

39. The composition of any one of claims 31-37, wherein the composition is formulated for intravenous delivery.


SEQUENCE LISTING (6 sequences)

SEQ ID NO: 1 - DNA, 1200 bp, synthetic construct
  source 1..1200: mol_type = other DNA
atgaaaatgcggttcttggggttggtggtctgtttggttctctggaccctgcattctgaggggtctggagggaaactgacagctgtggatcctgaaacaaacatgaatgtgagtgaaattatctcttactggggattccctagtgaggaatacctagttgagacagaagatggatatattctgtgccttaaccgaattcctcatgggaggaagaaccattctgacaaaggtcccaaaccagttgtcttcctgcaacatggcttgctggcagattctagtaactgggtcacaaaccttgccaacagcagcctgggcttcattcttgctgatgctggttttgacgtgtggatgggcaacagcagaggaaatacctggtctcggaaacataagacactctcagtttctcaggatgaattctgggctttcagttatgatgagatggcaaaatatgacctaccagcttccattaacttcattctgaataaaactggccaagaacaagtgtattatgtgggtcattctcaaggcaccactataggttttatagcattttcacagatccctgagctggctaaaaggattaaaatgttttttgccctgggtcctgtggcttccgtcgccttctgtactagccctatggccaaattaggacgattaccagatcatctcattaaggacttatttggagacaaagaatttcttccccagagtgcgtttttgaagtggctgggtacccacgtttgcactcatgtcatactgaaggagctctgtggaaatctctgttttcttctgtgtggatttaatgagagaaatttaaatatgtctagagtggatgtatatacaacacattctcctgctggaacttctgtgcaaaacatgttacactggagccaggctgttaaattccaaaagtttcaagcctttgactggggaagcagtgccaagaattattttcattacaaccagagttatcctcccacatacaatgtgaaggacatgcttgtgccgactgcagtctggagcgggggtcacgactggcttgcagatgtctacgacgtcaatatcttactgactcagatcaccaacttggtgttccatgagagcattccggaatgggagcatcttgacttcatttggggcctggatgccccttggaggctttataataaaattattaatctaatgaggaaatatcagtga

SEQ ID NO: 2 - protein, 399 aa, synthetic construct
MKMRFLGLVVCLVLWTLHSEGSGGKLTAVDPETNMNVSEIISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 3 - protein, 448 aa, synthetic construct
CCCTAAAATGGGCAAACATTGCAAGCAGCAAACAGCAAACACACAGCCCTCCCTGCCTGCTGACCTTGGAGCTGGGGCAGAGGTCAGAGACCTCTCTGGGCCCATGCCACCTCCAACATCCACTCGACCCCTTGGAATTTCGGTGGAGAGGAGCAGAGGTTGTCCTGGCGTGGTTTAGGTAGTGTGAGAGGGGAATGACTCCTTTCGGTAAGTGCAGTGGAAGCTGTACACTGCCCAGGCAAAGCGTCCGGGCAGCGTAGGCGGGCGACTCAGATCCCAGCCAGTGGACTTAGCCCCTGTTTGCTCCTCCGATAACTGGGGTGACCTTGGTTAATATTCACCAGCAGCCTCCCCCGTTGCCCCTCTGGATCCACTGCTTAAATACGGACGAGGACAGGGCCCTGTCTCCTCAGCTTCAGGCACCACCACTGACCTGGGACAGTGAATC

SEQ ID NO: 4 - DNA, 5932 bp, synthetic construct
  source 1..5932: mol_type = other DNA
acccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgtgtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgatcggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagcttccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactgaggtaccggccgctagccgccaccatgaaaatgcggttcttggggttggtggtctgtttggttctctggaccctgcattctgaggggtctggagggaaactgacagctgtggatcctgaaacaaacatgaatgtgagtgaaattatctcttactggggattccctagtgaggaatacctagttgagacagaagatggatatattctgtgccttaaccgaattcctcatgggaggaagaaccattctgacaaaggtcccaaaccagttgtcttcctgcaacatggcttgctggcagattctagtaactgggtcacaaaccttgccaacagcagcctgggcttcattcttgctgatgctggttttgacgtgtggatgggcaacagcagaggaaatacctggtctcggaaacataagacactctcagtttctcaggatgaattctgggctttcagttatgatgagatggcaaaatatgacctaccagcttccattaacttcattctgaataaaactggccaagaacaagtgtattatgtgggtcattctcaaggcaccactataggttttatagcattttcacagatccctgagctggctaaaaggattaaaatgttttttgccctgggtcctgtggcttccgtcgccttctgtactagccctatggccaaattaggacgattaccagatcatctcattaaggacttatttggagacaaagaatttcttccccagagtgcgtttttgaagtggctgggtacccacgtttgcactcatgtcatactgaaggagctctgtggaaatctctgttttcttctgtgtggatttaatgagagaaatttaaatatgtctagagtggatgtatatacaacacattctcctgctggaacttctgtgcaaaacatgttacactggagccaggctgttaaattccaaaagtttcaagcctttgactggggaagcagtgccaagaattattttcattacaaccagagttatcctcccacatacaatgtgaaggacatgcttgtgccgactgcagtctggagcgggggtcacgactggcttgcagatgtctacgacgtcaatatcttactgactcagatcaccaacttggtgttccatgagagcattccggaatgggagcatcttgacttcatttggggcctggatgccccttggaggctttataataaaattattaatctaatgaggaaatatcagtgagcatgcactagtgcggccgcggatctcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaggtttaaaccccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattcgttcttcggggcgaaaactctcaaggatcttaccgctgttgagatccagttcgatgtaacccactcgtgc

SEQ ID NO: 5 - DNA, 5932 bp, synthetic construct; identical to SEQ ID NO: 4
  source 1..5932: mol_type = other DNA

SEQ ID NO: 6 - DNA, 5932 bp, synthetic construct
  source 1..5932: mol_type = other DNA
tgggttgactagaagtcgtagaaaatgaaagtggtcgcaaagacccactcgtttttgtccttccgttttacggcgttttttcccttattcccgctgtgcctttacaacttatgagtatgagaaggaaaaagttataataacttcgtaaatagtcccaataacagagtactcgcctatgtataaacttacataaatctttttatttgtttatccccaaggcgcgtgtaaaggggcttttcacggtggactgcagattctttggtaataatagtactgtaattggatatttttatccgcatagtgctccgggaaagcagagcgcgcaaagccactactgccacttttggagactgtgtacgtcgagggcctctgccagtgtcgaacagacattcgcctacggccctcgtctgttcgggcagtcccgcgcagtcgcacacaaccgcccacagccccgaccgaattgatacgccgtagtctcgtctaacatgactctcacgtggtatacgccacactttatggcgtgtctacgcattcctcttttatggcgtagtccgctaaggttgtaggttatttagtatgtccgttccgtttcttaatcgttttaattcgttatttcggagtctcgtatttcgatttagccaacatggtttttgtaatactgggacattatgaaaacgccctcttcggaaataaagttgcgttcctatttttaaaaatcttgggagtatataaaatttacgttacggactcattacacatccatttctaagtttgcccactctttccggcctctgtcagtttagtggtagttatactataagttggcaagatcgactatttaagtacggcctctcccatcgataaaaactctccagagatgtttccgatagtccagtaacggactctcagacctcgtttgttctcttagctacttgccattagcattttgatcgtacagttagtatacatggggccaactattagtcttttcggggtttttgtccttctaacatattcgtttataaatttaacattcgcaattataaaacaattttaagcgcaatttaaaaacaatttagtcgagtaaaaaattggttatccggctttagccgttttagggaatatttagttttcttatctggctctatcccaactcacaacaaggtcaaaccttgttctcaggtgataatttcttgcacctgaggttgcagtttcccgctttttggcagatagtcccgctaccgggtgatgcacttggtagtgggattagttcaaaaaaccccagctccacggcatttcgtgatttagccttgggatttccctcgggggctaaatctcgaactgcccctttcggccgcttgcaccgctctttccttcccttctttcgctttcctcgcccgcgatcccgcgaccgttcacatcgccagtgcgacgcgcattggtggtgtgggcggcgcgaattacgcggcgatgtcccgcgcatgataccaacgaaactgctcgtgcatattgcacgaaaggagcaatcttagtctcgccctcgatttgtcctccggctaatttccctaaaatctgtccttgccatgcggtcttaggactcttcacaaaaatattagtcactccggtggctcattttctcagacaggtagtgcgtttaattggcaacagcgttatgaagaaactaatcattattgtagtgaacggactcatcttcttgagtttgatagccggaacgaccattataggtcttgttataatggcggtcggtaacgttgccttagcggtaagcggtaagtccgacgcgttgacaacccttcccgctagccacgcccggagaagcgataatgcggtcgacgcgcgagcgagcgagtgactccggcgggcccgtttcgggcccgcagcccgctggaaaccagcgggccggagtcactcgctcgctcgcgcgtctctccctcaccggttgaggtagtgatccccaaggatccttcgaagggattttacccgtttgtaacgttcgtcgtttgtcgtttgtgtgtcgggagggacggacgactggaacctcgaccccgtctccagtctctggagagacccgggtacggtggaggttgtaggtgagctggggaaccttaaagccacctctcctcgtctccaacaggaccgcaccaaatccatcacactctccccttactgaggaaagccattcacgtcaccttcgacatgtgacgggtccgtttcgcaggcccgtcgcatccgcccgctgagtctagggtcggtcacctgaatcggggacaaacgaggaggctattgaccccactggaaccaattataagtggtcgtcggagggggcaacggggagacctaggtgacgaatttatgcctgctcctgtcccgggacagaggagtcgaagtccgtggtggtgactggaccctgtcacttaggcctgagattccatttatattttaaaaattcacatattacacaatttgatgactaagattaacaaagagagaaaatctaaggttggaaaccttgactccatggccggcgatcggcggtggtacttttacgccaagaaccccaaccaccagacaaaccaagagacctgggacgtaagactccccagacctccctttgactgtcgacacctaggactttgtttgtacttacactcactttaatagagaatgacccctaagggatcactccttatggatcaactctgtcttctacctatataagacacggaattggcttaaggagtaccctccttcttggtaagactgtttccagggtttggtcaacagaaggacgttgtaccgaacgaccgtctaagatcattgacccagtgtttggaacggttgtcgtcggacccgaagtaagaacgactacgaccaaaactgcacacctacccgttgtcgtctcctttatggaccagagcctttgtattctgtgagagtcaaagagtcctacttaagacccgaaagtcaatactactctaccgttttatactggatggtcgaaggtaattgaagtaagacttattttgaccggttcttgttcacataatacacccagtaagagttccgtggtgatatccaaaatatcgtaaaagtgtctagggactcgaccgattttcctaattttacaaaaaacgggacccaggacaccgaaggcagcggaagacatgatcgggataccggtttaatcctgctaatggtctagtagagtaattcctgaataaacctctgtttcttaaagaaggggtctcacgcaaaaacttcaccgacccatgggtgcaaacgtgagtacagtatgacttcctcgagacacctttagagacaaaagaagacacacctaaattactctctttaaatttatacagatctcacctacatatatgttgtgtaagaggacgaccttgaagacacgttttgtacaatgtgacctcggtccgacaatttaaggttttcaaagttcggaaactgaccccttcgtcacggttcttaataaaagtaatgttggtctcaataggagggtgtatgttacacttcctgtacgaacacggctgacgtcagacctcgcccccagtgctgaccgaacgtctacagatgctgcagttatagaatgactgagtctagtggttgaaccacaaggtactctcgtaaggccttaccctcgtagaactgaagtaaaccccggacctacggggaacctccgaaatattattttaataattagattactcctttatagtcactcgtacgtgatcacgccggcgcctagagtctgtactattctatgtaactactcaaacctgtttggtgttgatcttacgtcactttttttacgaaataaacactttaaacactacgataacgaaataaacattggtaatattcgacgttatttgttcaattgttgttgttaacgtaagtaaaatacaaagtccaagtccccctccacaccctccaaaaaatccaaatttggggtgagggagagacgcgcgagcgagcgagtgactccggcccgctggtttccagcgggctgcgggcccgaaacgggcccgccggagtcactcgctcgctcgcgcgtcgacgtaattacttagccggttgcgcgcccctctccgccaaacgcataacccgcgagaaggcgaaggagcgagtgactgagcgacgcgagccagcaagccgacgccgctcgccatagtcgagtgagtttccgccattatgccaataggtgtcttagtcccctattgcgtcctttcttgtacactcgttttccggtcgttttccggtccttggcatttttccggcgcaacgaccgcaaaaaggtatccgaggcggggggactgctcgtagtgtttttagctgcgagttcagtctccaccgctttgggctgtcctgatatttctatggtccgcaaagggggaccttcgagggagcacgcgagaggacaaggctgggacggcgaatggcctatggacaggcggaaagagggaagcccttcgcaccgcgaaagagtatcgagtgcgacatccatagagtcaagccacatccagcaagcgaggttcgacccgacacacgtgcttggggggcaagtcgggctggcgacgcggaataggccattgatagcagaactcaggttgggccattctgtgctgaatagcggtgaccgtcgtcggtgaccattgtcctaatcgtctcgctccatacatccgccacgatgtctcaagaacttcaccaccggattgatgccgatgtgatcttcctgtcataaaccatagacgcgagacgacttcggtcaatggaagcctttttctcaaccatcgagaactaggccgtttgtttggtggcgaccatcgccaccaaaaaaacaaacgttcgtcgtctaatgcgcgtctttttttcctagagttcttctaggaaactagaaaagatgccccagactgcgagtcaccttgcttttgagtgcaattccctaaaaccagtacttgttattttgacagacgaatgtatttgtcattatgttccccacaatactcggtataagttgccctttgcagaacgagatccggcgctaatttaaggttgtacctacgactaaatatacccatatttacccgagcgctattacagcccgttagtccacgctgttagatagctaacatacccttcgggctacgcggtctcaacaaagactttgtaccgtttccatcgcaacggttactacaatgtctactctaccagtctgatttgaccgactgccttaaatacggagaaggctggtagttcgtaaaataggcatgaggactactacgtaccaatgagtggtgacgctaggggcccttttgtcgtaaggtccataatcttcttataggactaagtccacttttataacaactacgcgaccgtcacaaggacgcggccaacgtaagctaaggacaaacattaacaggaaaattgtcgctagcgcataaagcagagcgagtccgcgttagtgcttacttattgccaaaccaactacgctcactaaaactactgctcgcattaccgaccggacaacttgttcagacctttctttacgtatttgaaaacggtaagagtggcctaagtcagcagtgagtaccactaaagagtgaactattggaataaaaactgctcccctttaattatccaacataactacaacctgctcagccttagcgtctggctatggtcctagaacggtaggataccttgacggagccactcaaaagaggaagtaatgtctttgccgaaaaagtttttataccataactattaggactatacttatttaacgtcaaagtaaactacgagctactcaaaaagattcttaagcaagaagccccgcttttgagagttcctagaatggcgacaactctaggtcaagctacattgggtgagcacg